SEQUENCE LISTING (26 sequences)

SEQ ID NO: 1 - DNA, 3791 bp, Homo sapiens
cacccaagtctgagttgctaaaaaatggagctgtcactgggccttgctctgccaggacctgcagagccggggacctctctgtggcaagcccagcaagatgactgctctgaggcgccctagggctgagggaggggccgtgacaccagccccgccccccagccacctgggaaaaggaagcacaaaaaggagaagcagcaacggctgctctgcttccttcccatctcgctcttgggtcatgcctggccagcagaaagcagctccataggggaggagagccacgcaggatctcacagctgcagtctaatagtaacacagaggattcagcagtggccaccatgggttctgtgaattccagaggtcacaaggcggaagcccaggtggtgatgatgggcctggactcggcgggcaagaccacgctcctttacaagctgaagggccaccagctggtggagaccctgcccactgttggtttcaacgtggagcctctgaaagctcctgggcacgtgtcactgactctctgggacgttggggggcaggccccgctcagagccagctggaaggactatctggaaggcacagatatcctcgtgtacgtgctggacagcacagatgaagcccgcttacccgagtcggcggctgagctcacagaagtcctgaacgaccccaacatggctggcgtccccttcttggtgctggccaacaagcaggaggcacctgatgcacttccgctgcttaagatcagaaacaggctgagtctagagagattccaggaccactgctgggagctccggggctgcagtgccctcactggggaggggctgcccgaggccctgcagagcctgtggagcctcctgaaatctcgcagctgcatgtgtctgcaggcgagagcccatggggctgagcgcggagacagcaagagatcttgatccagacagagcagcatatctttgctcatacaaactagaagaaccagctgatccttgagaaatttacgcttagtctatcaaacaagaaatgctggcttggcccggtggctcatgcctgtaatcccagcactgtgggagaccacggtgggggaatcccttgagcccaggagttggagagcaacatcacaacaccccatttctactaataatcaaaaaattggccgggcatggtggcatgtgcctgtagtcccagctacttgggaggctgaggcaggagaatcgcttgagcccaagaggtagaggttgcagtgagccaagatcgcgccactgcactccagtctgggcaacagagtgagaccctgtctcaataataataataataataatgatgatactctaagaaaaaaatctcaacatacttcatttaatagctcgttaccaagtgtgaatgaagcaatatgtcataatagagtagccactggttgcataataatagagacctaaattctcaaatagggaaagaggttttaaaatcaaatttgaggccaggtgcagtggctcatgggcggaggagggcagattacttgaggctaggagttcaagaccagcctggccaacatggtgaaaccccatctctactgaaaatacaaaaattaggcatagtggtgcacgcctgcagtcccagctactcaggaggttgaggcagaagaatcgcttgaacccaggaagtggaggttgcagtgagccgagattgtgctgctgcactccagcctgggtgaaaaagacaggctgtgtctccaaaaagaaaaaaaaaagtcaaattcaaatatcatctggacatgtcacaatggatcgcggatccttatgagtgattttccccagtggcccctggggatgtgccactgtcactcagaagggcaagctaggcagggcccatccaacagcaggggtctgcaggttagacgttccctgccctgggacgctcacccctgggcaagaggctggaagttcacaccatccaaaatttatccttgttttttttctgatgctaattagcctctcccgattttatgacatcttgtgttgatctttttcaaaaactcattttcttttttttccttctcttttctccttcttgtagcacatatctttcgttaaagatcagatcaataaaatattttatttattcattaatttaacaaaaaaaacagagcatttagtttgtggcaaaaacactgagctttcgaatatgaatcatgtgctttaggtgggagttgtgaattctgaagatacagatgacagtgacgaatgccttctgtctcatgattgacagggaaaaggaaggttgaccatagcatcctagaaggctcatcaggtgatcattacctagcatccatgaagcacctgaaattatttgcaaaatgttacgctttggaccatttttccggggaaggagatccagaactttttaccagattttcaaagacatctgtgactcccaaaagttaacaatcactgatgtggttgttgtatccctcatccaaccccagaacactttctgtaatctgagttttttaatggcaagtggcctatatttagcacctgttctcatgttaaacagctctgaatgttagatattctttcttatcctggactggttctctctatctctggagtaatgcagtataaattggccatcagtaccctcctaaaatctgagatctgccaggcccctcttctaacaccaggttaggcatgcttggttatttccagtacttgtgagtcaacatgtttcaagacgctgtgttagacactagggatgcaaagatgaatgagataaggcctcaggcctcatggaaggtgagacagtaaagacattactcccataaaaatgtgaggagagagactcagttcagcaactgtttattctgtttattgagcacttacttggaccaagcactgtggtcttggtgttttacatagactgtctctaattctcacaactctgcaaaatatatatattcccattttataaaactacaaactgaggctcagagaaggtgtgacctcttgttgcttgaggcacagagttataaagtaacatatctggaatttgaaatgagatctgtttagggctaatgctgcatttttctacaacatcatgcctctagaaggtttaagctaggtaggctttcagccagcagacatgatggggagagccttctaataagagggaagagactgcttggaagcatgaagggaggtgtaagaaagataagtaagtcagtgtacttgcaacagaggcttgggatgaagggtgggtgaagttgacatcacgatagaaaacaaaactggaatgggagtttaggtccaatttgggcaaggttgtttgaatttcaataatcaggggtttgggtcaaggaagaaaaatcatgggacttgccatttaggaggataattttgtggtagtgtggaggtgaaataaagagaaaagggaaccttggagctgggaaggcaggaaaccggctagatgaccatcacacagcaaaggagggagtggaagagagatgagaaaattgagagctattattaagaaaaacagttgagagaggaagaatttgaagagggctcaagattttgagtccacatgacagaaggactggaatgccatgaactggagaaggtgagcgctgaagaaccaggatgggacggggctggaacagctgggttcagcttttgcagggtgggtacgtgtttggttatagctgctttcagattgttccattatctgtactcccaacaaccctgccggatatatttgttggctttcactcaaaaaaaaaaaaaaaaaaaaaaaaaaaaaaa

SEQ ID NO: 2 - protein, 196 aa, Homo sapiens
MGSVNSRGHKAEAQVVMMGLDSAGKTTLLYKLKGHQLVETLPTVGFNVEPLKAPGHVSLTLWDVGGQAPLRASWKDYLEGTDILVYVLDSTDEARLPESAAELTEVLNDPNMAGVPFLVLANKQEAPDALPLLKIRNRLSLERFQDHCWELRGCSALTGEGLPEALQSLWSLLKSRSCMCLQARAHGAERGDSKRS

SEQ ID NO: 3 - RNA, 24 nt, Artificial Sequence
  Ribozyme
cugaugaguccgcgaggacgaaac

SEQ ID NO: 4 - RNA, 26 nt, Artificial Sequence
  Ribozyme
ncugaugaguccgcgaggacgaaacn

SEQ ID NO: 5 - DNA, 25 bp, Artificial Sequence
  Oligonucleotide primer
ccatgggttctgtgaattccagagg

SEQ ID NO: 6 - DNA, 25 bp, Artificial Sequence
  Oligonucleotide primer
cagtggtcctggaatctctctagac

SEQ ID NO: 7 - DNA, 24 bp, Artificial Sequence
  Oligonucleotide primer
gccagcagaaagcagctccatagg

SEQ ID NO: 8 - DNA, 24 bp, Artificial Sequence
  Oligonucleotide primer
ttcaggaggctccacaggctctgc

SEQ ID NO: 9 - DNA, 23 bp, Artificial Sequence
  Oligonucleotide primer
gaggtatgtattgaaagaagagg

SEQ ID NO: 10 - DNA, 23 bp, Artificial Sequence
  Oligonucleotide primer
aacaaaacccaataacaactcca

SEQ ID NO: 11 - DNA, 21 bp, Artificial Sequence
  Oligonucleotide primer
cagaagacagtagctgatgtg

SEQ ID NO: 12 - DNA, 21 bp, Artificial Sequence
  Oligonucleotide primer
gagcaaagatatgctgctctg

SEQ ID NO: 13 - DNA, 23 bp, Artificial Sequence
  Oligonucleotide primer
gctgagtccagagagattccagg

SEQ ID NO: 14 - DNA, 20 bp, Artificial Sequence
  Oligonucleotide primer
tctcgcctgcagacacatgc

SEQ ID NO: 15 - DNA, 591 bp, Homo sapiens
atgggttctgtgaattccagaggtcacaaggcggaagcccaggtggtgatgatgggcctggactcggcgggcaagaccacgctcctttacaagctgaagggccaccagctggtggagaccctgcccactgttggtttcaacgtggagcctctgaaagctcctgggcacgtgtcactgactctctgggacgttggggggcaggccccgctcagagccagctggaaggactatctggaaggcacagatatcctcgtgtacgtgctggacagcacagatgaagcccgcttacccgagtcggcggctgagctcacagaagtcctgaacgaccccaacatggctggcgtccccttcttggtgctggccaacaagcaggaggcacctgatgcacttccgctgcttaagatcagaaacaggctgagtctagagagattccaggaccactgctgggagctccggggctgcagtgccctcactggggaggggctgcccgaggccctgcagagcctgtggagcctcctgaaatctcgcagctgcatgtgtctgcaggcgagagcccatggggctgagcgcggagacagcaagagatcttga

SEQ ID NO: 16 - DNA, 17 bp, Homo sapiens
agctcccagcagtggtc

SEQ ID NO: 17 - DNA, 17 bp, Homo sapiens
  misc_feature 7, 11: n = A,T,C or G
agctccnagcngtggtc

SEQ ID NO: 18 - DNA, 17 bp, Homo sapiens
agctcctagcagtggtc

SEQ ID NO: 19 - protein, 200 aa, Homo sapiens
MGNGLSDQTSILSNLPSFQSFHIVILGLDCAGKTTVLYRLQFNEFVNTVPTKGFNTEKIKVTLGNSKTVTFHFWDVGGQEKLRPLWKSYTRCTDGIVFVVDSVDVERMEEAKTELHKITRISENQGVPVLIVANKQDLRNSLSLSEIEKLLAMGELSSSTPWHLQPTCAIIGDGLKEGLEKLHDMIIKRRKMLRQQKKKR

SEQ ID NO: 20 - protein, 192 aa, Homo sapiens
MGNISSNISAFQSLHIVMLGLDSAGKTTVLYRLKFNEFVNTVPTIGFNTEKIKLSNGTAKGISCHFWDVGGQEKLRPLWKSYSRCTDGIIYVVDSVDVDRLEEAKTELHKVTKFAENQGTPLLVIANKQDLPKSLPVAEIEKQLALHELIPATTYHVQPACAIIGEGLTEGMDKLYEMILKRRKSLKQKKKR

SEQ ID NO: 21 - protein, 201 aa, Homo sapiens
MGNHLTEMAPTASSFLPHFQALHVVVIGLDSAGKTSLLYRLKFKEFVQSVPTKGFNTEKIRVPLGGSRGITFQVWDVGGQEKLRPLWRSYNRRTDGLVFVVDAAEAERLEEAKVELHRISRASDNQGVPVLVLANKQDQPGALSAAEVEKRLAVRELAAATLTHVQGCSAVDGLGLQQGLERLYEMILKRKKAARGGKKRR

SEQ ID NO: 22 - protein, 176 aa, Mus musculus
MGSVNSRGHKAEAQVVMMGLDSAGKTTILYKLKGNQLVDTLPTVGFNVEPLEAPGHVSLTLWDIGGQTQLRATWKDYLEGIDLLVYVLDSTDEARLPEAVAELKEVLEDPNMAGVPFLVLANKQEAPGALPLLEIRNRLGLEGFQKHCWELRACSALTGQGLQEALQSLLHLLKSR

SEQ ID NO: 23 - protein, 173 aa, Rattus norvegicus
MGSVNSRGHKAQVVMLGLDCAGKTTILYKLKGNRLVDTLPTVGFNVEPLEAPGHVSLTLWDIGGQTQLRATWKDYLEGIDLLVYVLDSTDEARLPEAVAELEEVLEDPNMAGVPFLVLANKQEAPDALPLLEIRNRLDLERFQDHCWELRACSALTGQGLQEARQSLLHLLRS

SEQ ID NO: 24 - protein, 176 aa, Danio rerio
MGAIKSKRFKKPPQVLIMGLDSAGKSTLMYRQLHGVIMQTSPTVGFNVATLQLNKKTSLTVWDIGGQDTMRPNWKYYLEGCKVLVFVVDSSDYARIGEAQKALKKILHDEHLKGVPLMVLANKKDLPNTMTIREVSTKLDLDTYTDRQWEIQACSAVKGLGLQQAFLSIAKLLQKA

SEQ ID NO: 25 - protein, 179 aa, Drosophila melanogaster
MGLLSLLRKLRPNPEKEARILLLGLDNAGKTTILKQLASEDITTVTPTAGFNIKSVAADGFKLNVWDIGGQWKIRPYWKNYFANTDVLIYVIDCTDRTRLPEAGSELFEMLMDNRLKQVPVLIFANKQDMPDAMSAAEVAEKMSLVQLQGRTWEIKACTAVDGTGLKEGMDWVCKNMKK

SEQ ID NO: 26 - protein, 205 aa, Arabidopsis thaliana
MGARFSRIAKRFLPKSKVRILMVGLDGSGKTTILYKLKLGEVVTTVPTIGFNLETVEYKGINFTVWDIGGQEKIRKLWRHYFQNAQGLIFVVDSSDSERLSEARNELHRILTDNELEGACVLVFANKQDSRNALPVAEVANKLGLHSLSKRCWLIQGTSAISGQGLYEGLEWLSTTIPNKPERSTSVSSFRSDSYERKLVRGPRY

(19) United States
(12) Patent Application Publication
(10) Pub. No.: US 2005/0266443 A1
(43) Pub. Date: Dec. 1, 2005

(54) Novel tumor suppressor gene and compositions and methods for making and using the same

(75) Inventors: Carlo M. Croce, Columbus, OH (US); George A. Calin, Columbus, OH (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Thomas Jefferson University

(21) Appl. No.: 11/093,746

(22) Filed: Mar. 30, 2005

Related U.S. Application Data
(63) Continuation-in-part of application No. PCT/US03/32270, filed on Oct. 10, 2003.
(60) Provisional application No. 60/417,842, filed on Oct. 11, 2002.

(51) Int. Cl. C12Q 1/68; C07H 21/04; C12P 21/06; C07K 14/525

(52) U.S. Cl. 435/6; 435/69.1; 435/320.1; 435/325; 530/351; 536/23.5

(57) ABSTRACT

The present invention relates to the identification and cloning of ARTS1, a novel tumor suppressor gene. The invention further encompasses isolated proteins encoded by ARTS1, methods of making and using the same, methods of diagnosing the presence of, or prediposition for, a cancer associated with a defective ARTS1 gene or gene product, and methods of treating or preventing cancers associated with a defective ARTS1 gene or gene product.

### RELATED APPLICATIONS

This application is a continuation-in-part of International Application No. PCT/US2003/032270, which designated the United States, was filed on Oct. 10, 2003, and was published in English, which claims the benefit of U.S. Provisional Application No. 60/417,842, filed on Oct. 11, 2002. The entire teachings of the above applications are incorporated herein by reference.

### GOVERNMENT SUPPORT

This invention was supported, in whole or in part, by a grant under Program Project Grant P01CA76259, P01CA81534, and P30CA56036 from the National Cancer Institute. The Government has certain rights in this invention.

### FIELD OF THE INVENTION

The invention relates to the identification and cloning of ARTS1, a novel tumor suppressor gene, and to methods of making and using the same. ARTS1 was originally designated ARLTS1, and may also be referred to as ARLS1.

### BACKGROUND OF THE INVENTION

Loss-of-function mutations in tumor suppressor genes (TSGs) play an essential role in the initiation and progression of human tumors, while inactivation by methylation seems to be important for tumor progression (Weinberg, R. A. Science 254, 1138-46 (1991), which is incorporated herein by reference). Chromosome 13 at band q14, where the retinoblastoma (RB1) gene (Marshall, C. J. Cell 64, 313-26 (1991), which is incorporated herein by reference) is located, is hemizygously or homozygously deleted in a diversity of hematopoietic and solid tumors (Bullrich, F. & Croce, C. M. In Chronic Lymphoid Leukemias (ed. Chenson, B. D.) 9-32 (Marcel Dekker, Inc., New York Bassel, 2001), which is incorporated herein by reference). Several reports presented evidence for a new tumor suppressor locus telomeric to the RB1 gene (Brown, A. G., Ross, F. M., Dunne, E. M., Steel, C. M. & Weir-Thompson, E. M. Nat Genet 3:67-72 (1993), Howthorn, L. A., Chapman, R., Oscier, D. & Cowell, J. K. Oncogene 8:1415-1419 (1993) and Liu, Y., et al., Proc Natl Acad Sci USA 90:8697-8701 (1993), which are each incorporated herein by reference). However, none of the genes in the region were found to be inactivated by either combination of deletion, mutations or promoter hypermethylation.

There is a need to identify and clone TSGs whose loss of function are associated with the initiation and progression of human tumors. There is a need to identify a TSG telomeric to the RB1 gene. There is a need to identify nucleic acids which can serve as probes or primers for the detection of the TSG. There is a need for genetic based therapeutics which can be delivered to function in cells with a TSG mutation. There is a need for isolated protein and for antibodies which specifically react to the protein. There is a need for assays, reagents and kits to identify compounds that can upregulate, enhance and/or compensate for inactivity of the TSG. There is a need to study and understand the mechanisms by which the TSG is involved in initiation and progression of tumors and for reagents useful in such studies. There is a need to identify new cancer therapeutics and for kits and methods of identifying such compounds.

The identification and cloning of new TSGs, whose loss of function are associated with the initiation and progression of human tumors, also will be useful in developing new assays for diagnosing whether a subject has, or is at risk for developing, cancer.

### SUMMARY OF THE INVENTION

The invention relates to isolated proteins comprising the amino acid sequence shown in SEQ ID NO:2.

The invention relates to isolated nucleic acid molecules that comprise nucleic acid sequences that encode a protein that has an amino acid sequence shown in SEQ ID NO:2.

The invention relates to isolated nucleic acid molecules that comprise SEQ ID NO:1 or a fragment thereof having at least 10 nucleotides.

The invention relates to a recombinant expression vector comprising a nucleic acid molecule comprising SEQ ID NO:1.

The invention relates to a host cell comprising a recombinant expression vector comprising a nucleic acid molecule that comprises SEQ ID NO:1.

The invention relates to an oligonucleotide molecule comprising a nucleotide sequence complimentary to a nucleotide sequence of at least 5 nucleotides of SEQ ID NO:1.

The invention relates to isolated antibodies that bind to an epitope on SEQ ID NO:2.

The invention relates to methods of identifying compounds that are processed by Caspase-1.

In particular embodiments, the invention is a method of diagnosing whether a subject has, or is at risk for developing, a cancer. In one embodiment, the method comprises analyzing the nucleotide sequence of an ARTS1 gene in a sample from a subject, and comparing this sequence with the nucleotide sequence of an ARTS1 gene in a control sample. In another embodiment, the method comprises assessing the ARTS1 gene copy number in cells of a sample from a subject. In still another embodiment, the method encompasses evaluating the DNA methylation status of one or more regions of the ARTS1 gene in a sample from a subject, and comparing the status with the DNA methylation status of the corresponding ARTS1 gene region(s) in a control sample. In a further embodiment, the method involves determining the expression level of one or more ARTS1 gene products in a sample from a subject, and comparing this level to the expression level of the corresponding gene product in a control sample.

The present invention also encompasses methods of preventing or treating a subject who has a cancer. In one embodiment, the method comprises administering an effective amount of an ARTS1 gene or gene product to a subject. In a particular embodiment, the method comprises delivering an ARTS1 gene or gene product into the cells of a subject who has, or is at risk for developing, a cancer. In another embodiment, the cancer is associated with a defective ARTS1 gene. In another embodiment, a wild-type ARTS1 gene construct is administered to a subject with a defective ARTS1 gene, and is used for targeted gene replacement therapy, whereby the wild-type ARTS1 gene of the construct replaces a defective ARTS1 gene in the subject's cells.

### BRIEF DESCRIPTION OF THE DRAWINGS

FIGS. 1A-C. Localization of ARTS1 tumor suppressor gene at 13q14. FIG. 1A: The position of genetic markers and positions of genes on the map is shown. FIG. 1B: A multiple alignment of human ARTS1 (SEQ ID NO:2) with human ARL proteins—ARL4 (SEQ ID NO. 19), ARL7 (SEQ ID NO. 20) and ARL4L (SEQ ID NO. 21). Several motifs presumably involved in nucleotide binding and hydrolysis (PM1, PM3, G2 and G3), characteristic of Ras-related GTPases27, are also present in ARTS1 as indicated. Furthermore, five additional amino acids, typical of the ARF subfamily (G2, N47, W74, R95 and G161), are all conserved in ARTS1. In the C-terminus, ARTS1 harbors fewer arginine or lysine residues than ARL4, ARL7 and ARL4L. The location of the Trp 149Stop mutation is indicated by an asterisk. FIG. 1C: A multiple alignment of human ARTS1 protein (Homo sapiens, labeled as “Homo”; SEQ ID NO. 2) with ARTS1-like proteins in other species. Sequences for mouse (Mus musculus, labeled as “Mus”; SEQ ID NO. 22), rat (Rattus norvegicus, labeled as “Rattus”; SEQ ID NO. 23), zebrafish (Danio rerio, labeled as “Danio”; SEQ ID NO. 24), fruit fly (Drosophila melanogaster, labeled as “Drosophila”; SEQ ID NO. 25) and the plant, Arabidopsis thaliana (labeled as “Arabidopsis”; SEQ ID NO. 26) are provided.

FIGS. 2A-C. ARTS1 mRNA expression and methylation analysis. FIG. 2A: Northern blotting of ARTS1 in cancer cell lines shows absent or reduced expression of ARTS1 in several cell lines. FIG. 2B: ARTS1 expression correlates with the level of methylation of the ARTS1 locus analyzed by Southern blotting of digested genomic DNA with Bg1II (“B”) alone or in combination with HpaII (“BH”). The combination Bg1II+MspI (“BM”) was used to determine the fragment length without respect to methylation. The presence or absence of ARTS1 expression is shown by “+” or “−”, respectively, and the restriction map (Bg1II-B-thick vertical lines, HpaII-thin vertical lines) is drawn at the bottom. The position of the ORF probe used is indicated by *. FIG. 2C: Correlation between ARTS1 expression analyzed by RT-PCR and CpG sites methylation analyzed by bisulfite sequencing in fresh tumors; white and black rectangles represent unmethylated and hypermethylated CpGs respectively, while gray rectangles represent partially methylated CpG sites. As a control, Epstein-Barr Virus transformed lymphoblastoid cell lines were used.

FIGS. 3A-D. ARTS1 suppresses tumorigenicity in A549 cells. FIG. 3A: A Northern blot showing restoration of ARTS1 expression following transfection of the minigene into A549 cells. ARLTS1-A, -B, and -C designate three different clones of A549 cells that have been transfected with a construct for expressing full-length ARTS1 protein. A549 pMV-7 denotes A549 cells that have been transfected with empty pMV-7 vector. A vector with ARLTS1-antisense sequence also is included as a control. FIG. 3B: Tumor formation in nude mice. The weight (mg) of tumors for the five analyzed clones, determined at the indicated times, are shown. The same results were obtained by measurement of tumor size. FIG. 3C: Example of tumorigenesis in nude mice at 8 weeks after subcutaneous injection of 10⁶ cells expressing the indicated construct. FIG. 3D: Colony growth in soft agar for the indicated clones (data at 21 days after plating 5×10⁴ cells).

FIG. 4. Analysis of ARTS1 expression in human tissues by Northern blotting reveals that ARTS1 is ubiquitously expressed.

FIG. 5. Mutation analysis in ARTS1 shows the presence of the germline polymorphism G446A (Trp149Stop). The presented sequences are in reverse orientation. For identification of the G446A (Trp149Stop) mutation, a rapid assay was developed using the MaeI site introduced by the mutation. DNA was amplified using primers MaeI-F1 (which contains a changed base from the wild-type sequence to destroy a constitutive MaeI site; SEQ ID NO:13) and MaeI-R1 (SEQ ID NO:14) (for sequences of the primers, see Table 4), purified using QIAquick PCR purification kit (QIAGEN) and digested with 2U of MaeI (Boehringer Manhiem, Germany). The amplification of a normal allele (SEQ ID NO. 16) gives rise to a single 138 bp product, while the mutant allele (SEQ ID NOs. 17 and 18) produces two bands of 106 and 32 bp. Note that the digestion has low efficiency and only partial digestion products were obtained. Digested PCR products were loaded on a 3% agarose gel and visualized using a UV imager. N=normal and T=tumor.

FIG. 6. Both wild-type ARTS1 and the truncated ARLTS1 proteins are localized in cytoplasm and nucleus. Subcellular localization using ARTS1-GFP fusion protein. 293 cells were transfected with pARTS1-gfp, pÄC-ARTS1-gfp (PARLTS1-Stop-gfp) and control plasmid, pEGFPN1. Bright field (left) and fluorescence images (right) of the same microscopy field are presented.

FIG. 7. The sequence of the cDNA of human ARTS1 (SEQ ID NO:1) is shown. The GenBank Accession number for the sequence is AF441378.

FIG. 8. The pedigree chart of an Italian CLL family, characterized by (i) 3 cases of CLL in 2 successive generations, (ii) the phenomenon of anticipation (i.e., earlier onset and more severe phenotype in successive generations) and (iii) a higher frequency of secondary tumors (e.g., lung carcinoma, kidney carcinoma, thyroid adenoma and essential thrombocythemia), is presented. Squares indicate males, circles signify females, solid symbols indicate affected individuals and a circle with a black point denotes an obligate carrier. Both the genotype with respect to the G446A ARTS1 mutation (e.g, heterozygous G/A, homozygous A/A and wild-type G/G), as well as the age at diagnosis, are shown. Specific cancers are designated using the following abbreviations: CLL=chronic lymphocytic leukemia; Lung=lung carcinoma; Tyr Ad=thyroid adenoma; Kidney=kidney carcinoma; and Ess Tb=Essential Thrombocytemia. Generations are designated by capital roman numerals, I-IV. Because of the absence of unaffected individuals without the mutation, this family is not suitable for an LOD score.

FIGS. 9A-D. ARTS1 suppresses tumorigenicity of A549 cells and functions as a tumor suppressor gene. FIG. 9A is a Northern blot showing ARTS1 mRNA levels and FIG. 9B is a Western blot showing ARTS1 protein levels in cells transfected with either ARTS1-FL constructs, encoding full-length ARTS1 protein, or ARTS1-Stop constructs, encoding the truncated ARTS1 Trp149Stop mutant. ARTS1 expression in both control cells transfected with empty vector (A549 pMV-7) and untransfected cells (A549) are also shown. FL1-4 and Stop1-3 refer to different samples of transfected cells. ARTS1 AS=ARTS1 antisense. FIGS. 9C and 9D show tumor formation in nude mice. In FIG. 9C, the weights (mg) of tumors for nine analyzed clones were determined at the indicated times. Similar results were obtained by measurement of tumor size (hatched ruler below tumors represents mm units), as shown in FIG. 9D.

FIG. 10. ARTS1-FL and ARTS1-Stop proteins have differing effects on induction of apoptosis and cause distinct gene expression signatures in A549 cells. FIG. 10A is a bar graph depicting flow cytometric data, showing the percentage of apoptotic cells in a population of transfected A549 cells, as determined using Caspase 3 Apoptosis assays. Differences in the percentage of apoptotic cells in populations of ARTS1-FL, ARTS1-Stop and A549 WT cells were statistically significant at day 6 (P<0.001 by Chi-square). FIG. 10B depicts Western blots showing the levels of various apoptotic proteins in ARTS1-FL and ARTS1-Stop transfected clones. Activation of the intrinsic apoptotic pathway, including increased expression of the “apoptosome” complex molecules, APAF-1 and pro-caspase-9, and the effector protein, PARP, was observed. Levels of pro-caspase-8 (expressed via the extrinsic apoptosis pathway) and pro-caspase-2 (expressed in stress-induced apoptotic cells) were not significantly affected by ARTS1-FL or ARTS1-Stop expression in A549 cells. Levels of actin protein were monitored as a control. In FIG. 10C, microarray analysis revealed distinct gene expression signatures for A549 cells expressing either ARTS1-FL or ARTS1-Stop. Cells expressing ARTS1-Stop displayed lower levels of proapoptotic transcripts (for example, e.g., BCL2L13, PDCD61P, ARF6, GRF2, RAB32 and RAP2C) than cells expressing the full-length ARTS1 protein. Such differences were statistically significant. A white box represents a gene that is underexpressed, a box with dots indicates a gene that is strongly underexpressed, a gray box represents a gene that is overexpressed, and a box with diagonal lines indicates a gene that is strongly overexpressed in comparison to the expression of the same gene in untransfected A549 cells. A black box indicates data not available.

### DETAILED DESCRIPTION OF THE INVENTION

The invention arises from the identification of human ARTS1, a novel member of the ADP-ribosylation factor family. ARTS1 is located at 13q14, and displays features characteristic of a tumor suppressor gene (TSG). ARTS1 is downregulated by DNA hypermethylation in 25 out of 75 (33%) human primary tumors and cell lines analyzed. Furthermore, analysis of 800 tumor and normal DNA samples revealed the presence of several ARTS1 variants, including a germline nonsense polymorphism G446A (Trp 149Stop), that is three times more frequent in cancer patients with a family history of cancer than in the normal population. Restoration of wild-type ARTS1 expression in A549 cells, which show low levels of ARTS1 expression, suppresses tumor formation in these cells.

The GenBank accession number of the human ARTS1 cDNA is AF441378. During the final stages of the functional studies described below, a clone of 1.6 kb, BC013150, containing the ORF of ARTS1 and encoding the hypothetical protein FLJ22595 (Accession Number AAH13150), was deposited with GenBank.

The ARTS1 gene, and proteins, polypeptides, or peptides encoded by the gene, can be used in methods of preventing abnormal cell growth in mammalian subjects. Such methods involve administering to a mammal a composition comprising an effective amount of an ARTS1 gene product (e.g., protein, RNA). Such methods also involve administering to a mammal a composition comprising an expression vector comprising a gene encoding ARTS1.

The discovery of ARTS1 provides the means to study its function as a TSG, to design probes and primers to detect its presence and/or to detect mutants, to prepare isolated nucleic acid molecules, to insert nucleic acid molecules that encode ARTS1 into vectors, such as cloning vectors to produce multiple copies, expression vectors useful to transform cells that will produce the protein, and gene therapy vectors which can be used to treat patients with tumors arising from a lack of endogenous ARTS1 function. Antisense compounds may be produced to generate tumor cells that lack ARTS1 function. These compounds can be used in assays to identify compounds useful to treat such cancers. Assays and kits can also be provided to identify compounds that upregulate or enhance ARTS1 activity. Transformed host cells may be used in methods to produce ARTS1 protein. Antibodies can be prepared that specifically bind to ARTS1 protein and used to isolate or detect the protein, including to distinguish wild-type ARTS1 from mutant ARTS1 proteins.

In certain embodiments, the present invention provides isolated ARTS1 protein that comprises the amino acid sequence shown in SEQ ID NO:2. The ARTS1 protein can be isolated from natural sources, produced by recombinant DNA methods or synthesized by standard protein synthesis techniques. In other embodiments, the invention relates to ARTS1-like polypeptides, which are polypeptides that are similar to, but differ from, the ARTS1 polypeptide by having at least one amino acid substitution, addition or deletion. For example, conservative amino acid substitutions may be made at one or more nonessential amino acid residues of the ARTS1 protein to generate ARTS1-like polypeptides. A “nonessential” amino acid residue is a residue that can be altered from the wild-type sequence of ARTS1 protein (e.g., the sequence of SEQ ID NO:2) without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Such substitutions would not be made for conserved amino acid residues, or for amino acid residues within a conserved motif.

The present invention also encompasses antibodies or antigen-binding fragments thereof that bind to ARTS1 polypeptides. The antibodies of the invention can be polyclonal or monoclonal, and the term “antibody” is intended to encompass both polyclonal and monoclonal antibodies. The terms polyclonal and monoclonal refer to the degree of homogeneity of an antibody preparation, and are not intended to be limited to particular methods of production. In one embodiment, the antibody or antigen-binding fragment is a monoclonal antibody or antigen-binding fragment thereof. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

The term “antibody” as used herein also encompasses functional fragments of antibodies, including fragments of chimeric, humanized, primatized, veneered or single chain antibodies. Functional fragments include antigen-binding fragments of antibodies that bind to an ARTS1 polypeptide (e.g., a mammalian ARTS1 polypeptide). For example, antibody fragments capable of binding to an ARTS1 polypeptide or a portion thereof, include, but are not limited to Fv, Fab, Fab′ and F(ab′)₂ fragments. Such fragments can be produced by enzymatic cleavage or by recombinant techniques. For example, papain or pepsin cleavage can generate Fab or F(ab′)₂ fragments, respectively. Other proteases with the requisite substrate specificity can also be used to generate Fab or F(ab′)₂ fragments. Antibodies can also be produced in a variety of truncated forms using antibody genes in which one or more stop codons have been introduced upstream of the natural stop site. For example, a chimeric gene encoding a F(ab′)₂ heavy chain portion can be designed to include DNA sequences encoding the CH₁ domain and hinge region of the heavy chain.

Single chain antibodies, and chimeric, humanized or primatized (CDR-grafted), or veneered antibodies, as well as chimeric, CDR-grafted or veneered single chain antibodies, comprising portions derived from different species, and the like are also encompassed by the present invention and the term “antibody”. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., Cabilly, et al., U.S. Pat. No. 4,816,567; Cabilly, et al., European Patent No. 0,125,023 B1; Boss, et al., U.S. Pat. No. 4,816,397; Boss, et al., European Patent No. 0,120,694 B1; Neuberger, M. S., et al., WO 86/01533; Neuberger, M. S., et al., European Patent No. 0,194,276 B1; Winter, U.S. Pat. No. 5,225,539; Winter, European Patent No. 0,239,400 B1; Queen, et al., European Patent No. 0 451 216 B1; and Padlan, E. A., et al., EP 0 519 596 A1. See also, Newman, R., et al., BioTechnology, 10: 1455-1460 (1992), regarding primatized antibody, and Ladner, et al., U.S. Pat. No. 4,946,778 and Bird, R. E., et al., Science, 242: 423-426 (1988)) regarding single chain antibodies.

Humanized antibodies can be produced using synthetic or recombinant DNA technology using standard methods or other suitable techniques. Nucleic acid (e.g., cDNA) sequences coding for humanized variable regions can also be constructed using PCR mutagenesis methods to alter DNA sequences encoding a human or humanized chain, such as a DNA template from a previously humanized variable region (see e.g., Kamman, M., et al., Nucl. Acids Res., 17: 5404 (1989)); Sato, K., et al., Cancer Research, 53: 851-856 (1993); Daugherty, B. L., et al., Nucleic Acids Res., 19(9): 2471-2476 (1991); and Lewis, A. P. and J. S. Crowe, Gene, 101: 297-302 (1991)). Using these or other suitable methods, variants can also be readily produced. In one embodiment, cloned variable regions can be mutated, and sequences encoding variants with the desired specificity can be selected (e.g., from a phage library; see e.g., Krebber, et al., U.S. Pat. No. 5,514,548; Hoogenboom, et al., WO 93/06213).

The antibody can be a humanized antibody comprising one or more immunoglobulin chains (e.g., an antibody comprising a CDR of nonhuman origin (e.g., one or more CDRs derived from an antibody of nonhuman origin) and a framework region derived from a light and/or heavy chain of human origin (e.g., CDR-grafted antibodies with or without framework changes)). In one embodiment, the antibody or antigen-binding fragment thereof comprises the light chain CDRs (CDR1, CDR2 and CDR3) and heavy chain CDRs (CDR1, CDR2 and CDR3) of a particular immunoglobulin. In another embodiment, the antibody or antigen-binding fragment further comprises a human framework region.

The antibodies described herein can also be conjugated to an agent. In one embodiment, the agent is a label, for example, a radioisotope, an epitope label (tag), an affinity label (e.g., biotin, avidin), a spin label, an enzyme, a fluorescent group or a chemiluminescent group. Labeled antibodies or antigen-binding fragments of the present invention can be used, e.g., in the diagnostic and/or prognostic methods described herein. In another embodiment, the antibody is conjugated to a drug, toxin or anti-inflammatory agent. Conjugation of a drug, toxin or anti-inflammatory agent to the anti-ARTS1 antibodies and antigen-binding fragments of the invention allows for targeting of these agents to sites of ARTS1 expression and/or activity. Drugs and toxins that can be conjugated to the antibodies of the present invention include, for example, chemotherapeutic agents (e.g., mitomycin C, paxlitaxol, methotrexate, 5-fluorouracil, cisplatin, cyclohexamide), toxins (e.g., ricin, gelonin), anti-inflammatory agents and other suitable agents.

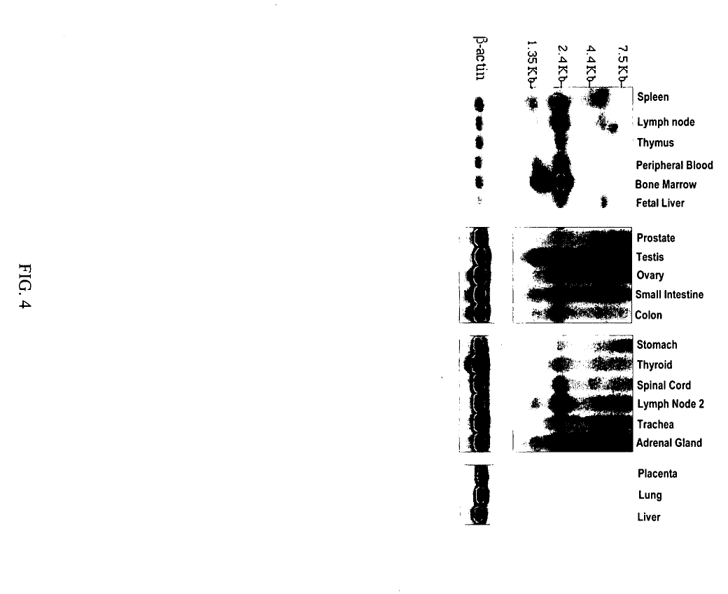

Antibodies that are specific for an ARTS1 polypeptide (e.g., a mammalian ARTS1 polypeptide) can be raised against an appropriate immunogen, such as an isolated and/or recombinant ARTS1 polypeptide or a portion thereof (including synthetic molecules, such as synthetic peptides). Antibodies can also be raised by immunizing a suitable host (e.g., mouse) with cells that express an ARTS1 polypeptide. One of skill in the art could readily identify such cells (see e.g., FIG. 4).

Preparation of immunizing antigen, and polyclonal and monoclonal antibody production can be performed using any suitable technique. A variety of methods have been described (see e.g., Kohler, et al., Nature, 256: 495-497 (1975) and Eur. J. Immunol. 6: 511-519 (1976); Milstein, et al., Nature 266: 550-552 (1977); Koprowski, et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer '94), Ausubel, F. M., et al., Eds., (John Wiley & Sons: New York, N.Y.), Chapter 11, (1991)). Generally, as exemplified herein, a hybridoma is produced by fusing a suitable immortal cell line (e.g., a myeloma cell line such as SP2/0, P3X63Ag8.653 or a heteromyeloma) with antibody-producing cells. Antibody-producing cells can be obtained from the peripheral blood or, preferably the spleen or lymph nodes, of humans or other suitable animals immunized with the antigen of interest. The fused cells (hybridomas) can be isolated using selective culture conditions, and cloned by limiting dilution. Cells that produce antibodies with the desired specificity can be selected by a suitable assay (e.g., ELISA).

Other suitable methods of producing or isolating antibodies of the requisite specificity (e.g., human antibodies or antigen-binding fragments) can be used, including, for example, methods that select recombinant antibody from a library (e.g., a phage display library). Transgenic animals capable of producing a repertoire of human antibodies (e.g., Xenomouse® (Abgenix, Fremont, Calif.)) can be produced using suitable methods (see e.g., Jakobovits, et al., Proc. Natl. Acad. Sci. USA, 90: 2551-2555 (1993); Jakobovits, et al., Nature, 362: 255-258 (1993)). Additional methods that are suitable for production of transgenic animals capable of producing a repertoire of human antibodies have been described (e.g., Lonberg, et al., U.S. Pat. No. 5,545,806; Surani, et al., U.S. Pat. No. 5,545,807; Lonberg, et al., WO97/13852).

In one embodiment, the antibody or antigen-binding fragment thereof has specificity for an ARTS1 polypeptide (e.g., a mammalian ARTS1 polypeptide). In a particular embodiment, the antibody or antigen-binding fragment thereof has specificity for a human ARTS1 polypeptide (e.g., such as depicted in SEQ ID NO:2). In another embodiment, the antibody or antigen-binding fragment thereof is an IgG or an antigen-binding fragment of an IgG. In another embodiment, the antibody or antigen-binding fragment thereof is an IgG1 or an antigen-binding fragment of an IgG1. In still other embodiments, the antibody or antigen-binding fragment thereof is an IgG2a, IgG2b, IgG3 antibody, or an antigen-binding fragment of any of the foregoing.

In one embodiment, the antibody is a human antibody or an antigen-binding fragment thereof. In another embodiment, the antibody is a humanized antibody or an antigen-binding fragment thereof. In yet another embodiment, the antibody or antigen-binding fragment can target another agent to a site of ARTS1 expression and/or activity in a cell. Such agents include, but are not limited to, therapeutic agents that enhance or promote one or more biological activities of ARTS1 protein, such as inhibition of tumorigenesis or cell death.

According to some embodiments, the present invention relates to an isolated nucleic acid molecule comprising a nucleotide sequence that encodes the amino acid sequence of SEQ ID NO:2. Such molecules can be routinely designed using the information set forth in SEQ ID NO:2. In certain embodiments, the invention relates to an isolated nucleic acid molecule comprising SEQ ID NO:1. Nucleic acid molecules that are fragments of nucleic acid molecules comprising a nucleotide sequence that encodes the amino acid sequence of SEQ ID NO:2, and of nucleic acid molecules comprising SEQ ID NO:1, also are encompassed by the present invention. By “fragment” is intended a portion of the nucleotide sequence encoding the ARTS1 protein, an ARTS1-like polypeptide, or a biologically-active fragment thereof. A fragment of an ARTS1 nucleotide sequence may encode a biologically-active portion of an ARTS1-like protein, or it may be a fragment that can be used as a hybridization probe or PCR primer. Methods of generating a biologically-active portion of an ARTS1-like protein are well known in the art. For example, a biologically-active portion of an ARTS1-like protein can be prepared by isolating a portion of one of the nucleotide sequences of the invention, expressing the encoded portion of the ARTS1-like protein (e.g., by recombinant expression in vitro), and assessing the activity of the encoded portion of the ARTS1-like protein (e.g., tumor suppressor or pro-apoptotic activities). A biologically-active portion of an ARTS1-like protein can also be generated by isolating or expressing a full-length ARTS1 protein or protein fragment and subjecting it to protease cleavage. Nucleic acid molecules that are fragments of an ARTS1′-like nucleotide sequence comprise at least about 10, 15, 20, 50, 75, 100, 200, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, or 1950, nucleotides, or up to the number of nucleotides present in a full-length ARTS1-like nucleotide sequence disclosed herein (for example, up to 3791 nucleotides of SEQ ID NO:1), depending upon the intended use. Nucleic acid molecules that are variants of the ARTS1 nucleotide sequences disclosed herein are also encompassed by the present invention. “Variants” of the ARTS1 nucleotide sequences include those sequences that encode the ARTS1 protein or ARTS1-like polypeptides disclosed herein, but that differ conservatively because of the degeneracy of the genetic code. These naturally-occurring allelic variants can be identified with the use of well-known molecular biology techniques, such as polymerase chain reaction (PCR) and hybridization techniques, as outlined below. Variant nucleotide sequences also include synthetically-derived nucleotide sequences that have been generated, for example, using site-directed mutagenesis, but which still encode ARTS1-like proteins. Generally, nucleotide sequence variants of the invention will have at least about 45%, 55%, 65%, 70%, 75%, 80%, 85%, 90%, 91%, 92%, 93%, 94%, 95%, 96%, 97%, 98%, or 99% identity to SEQ ID NO: 1. In one embodiment, the nucleotide sequence variant will encode an ARTS1 polypeptide or ARTS1′-like polypeptide having ARTS1 biological activity (e.g., tumor suppressor activity, pro-apoptotic activity).

The percent identity of two amino acid sequences (or two nucleic acid sequences) can be determined by aligning the sequences for optimal comparison purposes (e.g., gaps can be introduced in the sequence of a first sequence). The amino acids or nucleotides at corresponding positions are then compared, and the percent identity between the two sequences is a function of the number of identical positions shared by the sequences (i.e., % identity=# of identical positions/total # of positions×100). In certain embodiments, the length of the ARTS1 polypeptide, aligned for comparison purposes, is at least 30%, preferably, at least 40%, more preferably, at least 60%, and even more preferably, at least 70%, 80%, 90%, or 100%, of the length of the reference sequence, for example, the sequences described herein corresponding to an ARTS1 polypeptide (e.g., SEQ ID NO:2). The actual comparison of the two sequences can be accomplished by well-known methods, for example, using a mathematical algorithm. A preferred, non-limiting example of such a mathematical algorithm is described in Karlin, et al. Proc. Natl. Acad. Sci. USA, 90:5873-5877 (1993). Such an algorithm is incorporated into the BLASTN and BLASTX programs (version 2.2) as described in Schaffer, et al. Nucleic Acids Res., 29:2994-3005 (2001). When utilizing BLAST and Gapped BLAST programs, the default parameters of the respective programs (e.g., BLASTN; available at the Internet site for the National Center for Biotechnology Information) can be used. In one embodiment, the database searched is a non-redundant (NR) database, and parameters for sequence comparison can be set at: no filters; Expect value of 10; Word Size of 3; the Matrix is BLOSUM62; and Gap Costs have an Existence of 11 and an Extension of 1.

Another non-limiting example of a mathematical algorithm utilized for the comparison of sequences is the algorithm of Myers and Miller, CABIOS (1989). Such an algorithm is incorporated into the ALIGN program (version 2.0), which is part of the GCG (Accelrys, San Diego, Calif.) sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti (Comput. Appl. Biosci., 10: 3-5, 1994); and FASTA described in Pearson and Lipman (Proc. Natl. Acad. Sci USA, 85: 2444-2448, 1988).

In another embodiment, the percent identity between two amino acid sequences can be accomplished using the GAP program in the GCG software package (Accelrys, San Diego, Calif.) using either a Blossom 63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the GCG software package (Accelrys, San Diego, Calif.), using a gap weight of 50 and a length weight of 3.

Using standard techniques and readily available starting materials, a nucleic acid molecule that encodes ARTS1 may be isolated from a cDNA library, using probes or primers which are designed using the nucleotide sequence information disclosed in SEQ ID NO:1. In some embodiments, the nucleic acid molecules comprise the nucleotide sequence that consists of the coding sequence in SEQ ID NO:1. In some embodiments, the nucleic acid molecules consist of the nucleotide sequence set forth in SEQ ID NO:1. The isolated nucleic acid molecules of the invention are useful to prepare constructs and recombinant expression systems for preparing ARTS1.

A cDNA library may be generated by well-known techniques. A cDNA clone that contains one of the nucleotide sequences disclosed herein is identified using probes that comprise at least a portion of the nucleotide sequence disclosed in SEQ ID NO:1. The probes have at least 16 nucleotides, preferably 24 nucleotides. The probes can be used to screen a cDNA library using standard hybridization techniques. Alternatively, genomic clones may be isolated using genomic DNA from any mammalian (e.g., human) cell as a starting material. In certain embodiments, the present invention relates to isolated nucleic acid molecules that comprise a nucleotide sequence identical or complementary to a fragment of SEQ ID NO:1 which is at least 10 nucleotides. In some embodiments, the isolated nucleic acid molecules consist of a nucleotide sequence that is identical or complementary to a fragment of SEQ ID NO:1 that is at least 10 nucleotides. In some embodiments, the isolated nucleic acid molecules comprise, or consist of, a nucleotide sequence that is identical or complementary to a fragment of SEQ ID NO:1 that is 15-150 nucleotides. In some embodiments, the isolated nucleic acid molecules comprise, or consist of, a nucleotide sequence identical or complementary to a fragment of SEQ ID NO:1 that is 15-30 nucleotides. Isolated nucleic acid molecules that comprise, or consist of, a nucleotide sequence identical or complementary to a fragment of SEQ ID NO:1 that is at least 10 nucleotides are useful, e.g., as (1) probes for identifying genes and cDNA sequences comprising SEQ ID NO:1, (2) PCR primers for amplifying genes and cDNA comprising SEQ ID NO:1, and (3) antisense molecules for inhibiting transcription and translation of genes and cDNA, respectively, which encode ARTS1.

The ARTS1 nucleic acids of the invention may be used as molecular markers in electrophoresis assays in which the ARTS1 nucleic acid from a sample is separated on an electrophoresis gel and ARTS1 probes are used to identify bands which hybridize to such probes. Specifically, SEQ ID NO:1 or portions thereof, may be used as a molecular marker in electrophoresis assays in which the ARTS1 nucleic acid from a sample is separated on an electrophoresis gel and ARTS1-specific probes are used to identify bands that hybridize to them, indicating that the bands have a nucleotide sequence complementary to the sequence of the probes. The isolated nucleic acid molecule provided as a size marker will appear as a positive band that is known to hybridize to the probes and thus, can be used as a reference point for the size of the nucleic acid that encodes ARTS1. Electrophoresis gels useful in such an assay include standard polyacrylamide gels as described in Sambrook, et al., Molecular Cloning a Laboratory Manual, Second Ed. Cold Spring Harbor Press (1989), which is incorporated herein by reference.

The nucleotide sequences in SEQ ID NO:1 may be used to design probes, primers and complementary molecules that specifically hybridize to the unique nucleotide sequences of ARTS1. Probes, primers and complementary molecules that specifically hybridize to a nucleotide sequence that encodes ARTS1 may be designed routinely by those having ordinary skill in the art. As used herein, the term “specifically hybridize to nucleotide sequence that encodes ARTS1” is meant to refer to nucleic acid molecules with unique nucleotide sequences that hybridize to ARTS1 encoding sequences (e.g., sequences identical to portions of SEQ ID NO:1), but not other known protein encoding sequences. Thus, the unique sequences described herein are those that do not overlap with known sequences.

The present invention also includes labeled oligonucleotides that are useful as probes for performing oligonucleotide hybridization methods to identify ARTS1. The oligonucleotides include sequences that specifically hybridize to nucleotide sequences that encode ARTS1. Accordingly, the present invention includes probes that can be labeled and hybridized to unique nucleotide sequences that encode ARTS1. The labeled probes of the present invention can be labeled with radiolabeled nucleotides or can be otherwise detectable by readily available nonradioactive detection systems. In some preferred embodiments, probes comprise oligonucleotides consisting of from 10 to 100 nucleotides. In some embodiments, probes comprise oligonucleotides consisting of from 10 to 50 nucleotides. In other embodiments, probes comprise oligonucleotides consisting of from 12 to 20 nucleotides. The probes preferably contain nucleotide sequence completely identical or complementary to a fragment of a unique nucleotide sequence of ARTS1.

PCR technology is practiced routinely by those having ordinary skill in the art and its uses in diagnostics are well known and accepted. Methods for practicing PCR technology are disclosed in “PCR Protocols: A Guide to Methods and Applications”, Innis, M. A., et al., Eds. Academic Press, Inc. San Diego, Calif. (1990), which is incorporated herein by reference. Applications of PCR technology are disclosed in “Polymerase Chain Reaction” Erlich, H. A., et al., Eds. Cold Spring Harbor Press, Cold Spring Harbor, N.Y. (1989), which is incorporated herein by reference. Some simple rules aid in the design of efficient primers. Typical primers are 18-28 nucleotides in length having 50% to 60% g+c composition. The entire primer is preferably complementary to the sequence it must hybridize to. Preferably, primers generate PCR products having from 100 base pairs to 2000 base pairs. However, it is possible to generate products of 50 base pairs to up to 10 kb and more.

PCR technology allows for the rapid generation of multiple copies of nucleotide sequences by providing 5′ and 3′ primers that hybridize to sequences present in a nucleic acid molecule, and further providing free nucleotides and an enzyme which fills in the complementary bases to the nucleotide sequence between the primers with the free nucleotides to produce a complementary strand of DNA. The enzyme will fill in the complementary sequences adjacent to the primers. If both the 5′ primer and 3′ primer hybridize to nucleotide sequences on the complementary strands of the same fragment of nucleic acid, exponential amplification of a specific double-stranded product results. If only a single primer hybridizes to the nucleic acid molecule, linear amplification produces single-stranded products of variable length. PCR primers include at least one primer which includes a nucleotide sequence that specifically hybridizes to nucleotide sequence that encodes ARTS1.

One having ordinary skill in the art can isolate the nucleic acid molecule that encodes ARTS1 and insert it into an expression vector using standard techniques and readily available starting materials.

The present invention relates to a recombinant expression vector that comprises a nucleotide sequence that encodes an ARTS1 protein that comprises the amino acid sequence of SEQ ID NO:2. As used herein, the term “recombinant expression vector” is meant to refer to a plasmid, phage, viral particle or other vector, which, when introduced into an appropriate host, contains the necessary genetic elements to direct expression of the sequence that encodes the ARTS1 protein of the invention. This coding sequence is operably linked to necessary regulatory sequences. Expression vectors are well known and readily available. Examples of expression vectors include plasmids, phages, viral vectors and other nucleic acid molecules or nucleic acid molecule-containing vehicles that are useful to transform host cells and facilitate expression of coding sequences. In some embodiments, the recombinant expression vector comprises the nucleotide sequence set forth in SEQ ID NO:1. The recombinant expression vectors of the invention are useful for transforming hosts to prepare recombinant expression systems for preparing ARTS1.

The present invention relates to a host cell that comprises the recombinant expression vector comprising a nucleotide sequence that encodes an ARTS1 gene comprising SEQ ID NO:1. In some embodiments, the host cell comprises a recombinant expression vector that comprises SEQ ID NO:1. Host cells for use in well known recombinant protein expression systems are well known and readily available. Examples of host cells include bacterial cells such as E. coli, yeast cells such as S. cerevisiae, insect cells such as S. frugiperda, non-human mammalian tissue culture cells such as chinese hamster ovary (CHO) cells, and human tissue culture cells, such as HeLa cells.

The present invention relates to a transgenic non-human mammal that comprises the recombinant expression vector comprising a nucleic acid sequence that encodes an ARTS1 protein comprising the amino acid sequence of SEQ ID NO:2. Transgenic non-human mammals useful to produce recombinant proteins are well known, as are expression vectors and techniques necessary for generating transgenic animals. Generally, the transgenic animal comprises a recombinant expression vector in which the nucleotide sequence that encodes ARTS1 protein is operably-linked to a mammary cell-specific promoter whereby the coding sequence is only expressed in mammary cells and the recombinant protein is recovered from the animal's milk. In some embodiments, the sequence that encodes ARTS1 is SEQ ID NO:1.

In some embodiments, for example, one having ordinary skill in the art can, using well known techniques, insert such DNA molecules into a commercially available expression vector for use in well known expression systems. For example, the commercially available plasmid pSE420 (Invitrogen, San Diego, Calif.) may be used for production in E. coli. The commercially available plasmid, pYES2 (Invitrogen, San Diego, Calif.), may, for example, be used for production in S. cerevisiae strains of yeast. The commercially available MAXBAC™ complete baculovirus expression system (Invitrogen, San Diego, Calif.) may, for example, be used for production in insect cells. The commercially available plasmid, pcDNA I (Invitrogen, San Diego, Calif.), may, for example, be used for recombinant protein production in mammalian cells, such as Chinese Hamster Ovary cells. One having ordinary skill in the art can use these commercial expression vectors and systems, or others, to produce ARTS1 using routine techniques and readily available starting materials. (See e.g., Sambrook, et al., Molecular Cloning a Laboratory Manual, Second Ed. Cold Spring Harbor Press (1989) which is incorporated herein by reference.) Thus, the desired proteins can be prepared in both prokaryotic and eukaryotic systems, resulting in a spectrum of processed forms of the protein.

One having ordinary skill in the art may use other commercially available expression vectors and systems or produce vectors using well known methods and readily available starting materials. Expression systems containing the requisite control sequences, such as promoters and polyadenylation signals, and preferably enhancers, are readily available and known in the art for a variety of hosts (see e.g., Sambrook, et al., Molecular Cloning a Laboratory Manual, Second Ed. Cold Spring Harbor Press (1989)).

A wide variety of eukaryotic hosts are also now available for production of recombinant foreign proteins. As in bacteria, eukaryotic hosts may be transformed with expression systems that produce the desired protein directly. However, more commonly, signal sequences are provided to effect the secretion of the protein. Eukaryotic systems have the additional advantage of being able to process introns in the genomic sequences encoding many proteins of higher organisms. Eukaryotic systems also provide a variety of processing mechanisms which result in, for example, glycosylation, carboxy-terminal amidation, oxidation or derivatization of certain amino acid residues, conformational control, and so forth.

Commonly used eukaryotic systems include, but are not limited to, yeast, fungal cells, insect cells, mammalian cells, avian cells, and cells of higher plants. Suitable promoters are available, which are compatible and operable for use in each of these host types, as well as are termination sequences and enhancers, e.g., the baculovirus polyhedron promoter. As above, promoters can be either constitutive or inducible. For example, in mammalian systems, the mouse metallothionein promoter can be induced by the addition of heavy metal ions.

The particulars for the construction of expression systems suitable for desired hosts are known to those in the art. Briefly, for recombinant production of the protein, the DNA encoding the polypeptide is suitably ligated into the expression vector of choice. The DNA is operably linked to regulatory elements which are necessary for expression of the DNA in the selected host. One having ordinary skill in the art can, using well known techniques, prepare expression vectors for recombinant production of the polypeptide.

The expression vector including the DNA that encodes ARTS1 is used to transform the compatible host, which is then cultured and maintained under conditions that promote expression of the foreign DNA. The protein of the present invention, thus produced, is recovered from the culture, either by lysing the cells, or from the culture medium using techniques known to those in the art. One having ordinary skill in the art can, using well known techniques, isolate ARTS1 that is produced using such expression systems. The methods of purifying ARTS1 from natural sources, using antibodies which specifically bind to ARTS1, as described herein, may be equally applied to purifying ARTS1 produced by recombinant DNA methodology.

Examples of genetic constructs include ARTS1 coding sequence operably linked to a promoter that is functional in the cell line into which the constructs are transfected. Examples of constitutive promoters include promoters from cytomegalovirus or SV40. Examples of inducible promoters include mouse mammary leukemia virus or metallothionein promoters. Those having ordinary skill in the art can readily produce genetic constructs useful for transfecting cells with DNA that encodes ARTS1, using readily available starting materials. Such gene constructs are useful for the production of ARTS1.

In some embodiments of the invention, transgenic non-human animals are generated. The transgenic animals according to the invention contain the nucleic acids described herein (e.g., SEQ ID NO:1) under the regulatory control of a mammary-specific promoter. One having ordinary skill in the art, using standard techniques, such as those taught in U.S. Pat. No. 4,873,191, issued Oct. 10, 1989 to Wagner, and U.S. Pat. No. 4,736,866, issued Apr. 12, 1988 to Leder, both of which are incorporated herein by reference, can produce transgenic animals which produce ARTS1. Preferred animals are rodents, particularly rats and mice, and goats.

In addition to producing these proteins by recombinant techniques, automated peptide synthesizers may also be employed to produce ARTS1. Such techniques are well known to those having ordinary skill in the art and are useful if derivatives have substitutions not provided in DNA-encoded protein production.

In certain embodiments, the present invention is drawn to therapeutic methods that comprise administering an ARTS1 gene or gene product to a subject in need thereof. An ARTS1 gene can be any ARTS1 gene or fragment thereof that encodes a functional gene product. As used herein, a “functional gene product” is any gene product having one or more biological activities that are characteristic of ARTS1 polypeptides (e.g., tumor suppressor or pro-apoptotic activities). ARTS1 gene products include, but are not limited to, RNA and protein. In one embodiment, the gene product is a functional ARTS1 protein or fragment thereof. As used herein, a “functional ARTS1 protein” is one that is capable of carrying out one or more biological activities that are characteristic of ARTS1 polypeptides (e.g., tumor suppressor or pro-apoptotic activites).

One aspect of the invention relates to gene therapy, specifically “gene replacement.” Gene replacement” refers to the replacement of a mutated gene with a normal gene. The present invention provides methods of gene therapy, wherein the gene therapy is “gene replacement” therapy. Generally the present gene replacement method involves inhibition of an abnormal ARTS1 product, coupled with replacement with the normal ARTS1 gene. Generally, methods of the present invention can be used to treat conditions associated with tumorigenesis related to a lack of, or insufficient amount of, functional wild-type ARTS1. Methods of the present invention may be used to replace the abnormal ARTS1 gene with a normal ARTS1 gene.

By normal ARTS1 gene is meant any gene which, when encoded produces a biologically active, wild-type, tumor suppressing ARTS1 protein. By abnormal or mutant gene is meant any gene which, when encoded, does not produce a biologically active, wild-type ARTS1 protein and/or is insufficiently present to perform a tumor suppression function.

The term “DNA construct”, as used herein, refers to any DNA molecule which has been modified such that the nucleotide sequences in the molecule are not identical to a sequence which is produced naturally.

The term “expression vector”, as used herein, is defined as a DNA construct which includes an autonomous site of replication, a site of transcription initiation, and at least one structural gene coding for a protein that is to be expressed in a host organism. The expression vector will usually also contain appropriate control regions, such as a promoter and terminator, that control the expression of the protein in the host organism. Expression vectors of the present invention may include retroviral vectors, such as the “double copy” vector. As one skilled in the art would recognize, the choice of a particular vector depends partly upon the cell type that is targeted.

In preferred embodiments of the present invention, the expression vector includes a promoter. Vectors encoding one or more ribozymes should preferably utilize a strong, RNA polymerase III-type promoter. Useful promoters include, but are not limited to, tRNA and SV40 promoters. Expression vectors of the present invention may also include sequences homologous with those of a host gene, to provide for integration of the modified gene into a chromosome of the host.

The term “bifunctional expression vector”, as used herein, is defined as an expression vector that contains at least one structural gene cassette coding for a protein that is to be expressed in a host organism, and a regulatory cassette coding for one or more regulatory elements. The regulatory cassette may code for any element which functions within the cell to inhibit expression of one or more genes. In accordance with some embodiments of the present invention, the regulatory cassette codes for an RNA fragment having ribozyme activity effective to cleave a separate RNA molecule.

“Cassette”, as used herein, refers to a discrete DNA fragment that encodes a control region and a DNA sequence of interest, such a structural protein.

The term “plasmid” is used herein in accordance with its commonly accepted meaning, i.e., autonomously replicating, usually close-looped, DNA.

“Ribozyme” as the term is used herein, refers to an enzyme that is made of RNA. Ribozymes are involved in the cleavage and/or ligation of RNA chains. In preferred embodiments of the present invention, “hammerhead ribozymes” are used. As described above, hammerhead ribozymes cleave the phosphodiester bond of a target RNA downstream of a GUX triplet where X can be C, U, or A. Hammerhead ribozymes used in methods of the present invention have a structural domain having the sequence 5′-CUGAUGAGUCCGCGAGGACGAAAC-3′ (SEQ ID NO:3). Site specific regulatory elements, such as site specific ribozymes, are provided in accordance with the present invention. The ribozyme regulatory element is made site-specific, having the sequence 5′-Y-CUGAUGAGUCCGCGAGGACGAAAC-N-3′ (SEQ ID NO:4), where N and Y are complementary to regions of the target mRNA flanking the structural domain and are generally from about 20 to about 35 RNA bases in length, but need not be of equal lengths. However, it is preferable that neither is less than about 10 nucleotides.

Hammerhead ribozymes target the triplet, GUC. For a gene of interest, a target site can be identified by analyzing the gene sequence to identify GUC triplets. Computer analysis of secondary structure may assist in site selection. Denman, (1993), Biotechniques, 15, 1090-1094.

Vectors of the present invention may be delivered to a patient via methods known in the art. Retroviral-mediated delivery is preferred in some embodiments of the invention. In vivo delivery, by way of retroviral vectors, may be achieved, for example, by intravenous injection of the retroviral vectors. A double balloon catheter also may be used for direct delivery of retroviral vectors to the patient.

According to one aspect of the invention, compounds may be screened to identify compounds that inhibit or enhance Caspase-1 activity. Substrates of Caspase-1 include baculovirus protein p35 and the Sf immunophillin FKBP46. Assays may be performed by combining Caspase-1 with a substrate in the presence or absence of a test compound. The level of Caspase-1 activity in the presence of the test compound is compared to the level in the absence of the test compound. If Caspase-1 activity is increased by the presence of the test compound, the test compound is an enhancer. If Caspase-1 activity is decreased by the presence of the test compound, the test compound is an inhibitor. In some embodiments of the invention, the preferred concentration of test compound is from 1 μM to 500 μM. Another preferred concentration is from 10 μM to 100 μM. In some preferred embodiments, it is desirable to use a series of dilutions of test compounds.

Kits are included, which comprise containers with reagents necessary to screen test compounds. Such kits include a container with Caspase-1 protein, a container with a substrate such as FKBP46 or p35, which is preferably a labeled substrate, and instructions for performing the assay. Kits may include a control inhibitor, such as anti-Caspase-1 neutralizing antibodies.

Combinatorial libraries may be screened to identify compounds that enhance or inhibit Caspase-1 activity.

The present invention also provides methods of screening for, or diagnosing, whether a subject has, or is at risk for developing, a cancer by comparing an ARTS1 gene in a sample from a subject to an ARTS1 gene in a control sample from an unaffected individual. In one embodiment, an ARTS1 gene in a sample from a subject is screened for alterations in the nucleotide sequence of the gene relative to an ARTS1 gene in a control sample. An alteration of the nucleotide sequence of the ARTS1 gene in the sample, relative to a control sample, is indicative of the subject having, or being at risk for developing, cancer. As used herein, an alteration in the nucleotide sequence of an ARTS1 gene is any change affecting one or more nucleotides in the ARTS1 gene. Such changes include, but are not limited to, mutations, polymorphisms, deletions, translocations, insertions, inversions or any combination thereof. As used herein, a control sample is a sample from a subject who has a wild-type ARTS1 gene or a conservative allelic variant thereof.

In the practice of the present method, an alteration in the nucleotide sequence of an ARTS1 gene can be detected using any technique suitable for determining the structure or sequence of a gene in cells from a biological sample. For example, the presence of an ARTS1 gene alteration can be detected by Southern blot hybridization of the genomic DNA from a subject, using nucleic acid probes specific for ARTS1 gene sequences.

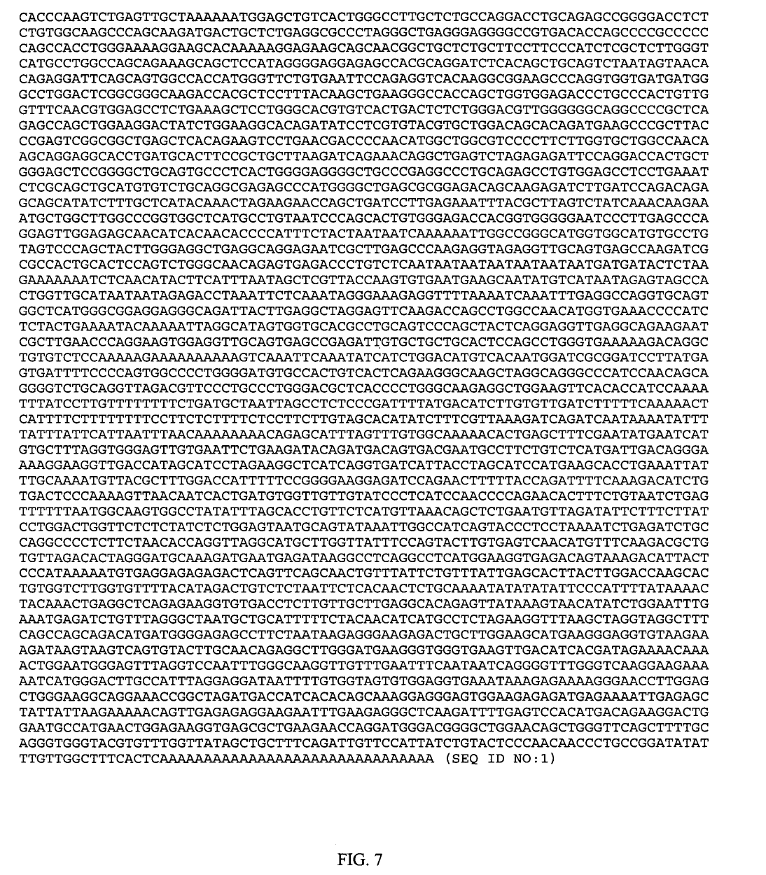

Southern blot hybridization techniques are within the skill in the art and are exemplified herein. For example, genomic DNA isolated from a subject's sample can be digested with restriction endonucleases. This digestion generates restriction fragments of the genomic DNA that can be separated by electrophoresis, for example, on an agarose gel. The restriction fragments are then blotted onto a hybridization membrane (e.g., nitrocellulose or nylon), and hybridized with labeled probes specific for the ARTS1 gene. A deletion or mutation of this gene is indicated by an alteration of the restriction fragment patterns on the hybridization membrane, as compared to DNA from a control sample that has been treated identically to the DNA from the subject's sample. Probe labeling and hybridization conditions suitable for detecting alterations in gene structure or sequence can be readily determined by one of ordinary skill in the art. The ARTS1 nucleic acid probes for Southern blot hybridization can be designed based upon the nucleic acid sequence provided in FIG. 7 (SEQ ID NO. 1), as described herein. Nucleic acid probe hybridization can then be detected by exposing hybridized filters to photographic film, or by employing computerized imaging systems, such as the Molecular Dynamics 400-B 2D Phosphorimager available from Amersham Biosciences, Piscataway, N.J.

An alteration in the nucleotide sequence of an ARTS1 gene can also be detected by amplifying a fragment of this gene using polymerase chain reaction (PCR), and analyzing the amplified fragment by direct sequencing or electrophoresis to determine if the sequence and/or length of the amplified fragment from the subject's DNA sample is different from that of a control DNA sample. Suitable reaction and cycling conditions for PCR amplification of DNA fragments can be readily determined by one of ordinary skill in the art.

In another embodiment, the ARTS1 gene copy number in cells from a biological sample from a subject is determined. A copy number that is less than two is indicative of the subject having, or being at risk for developing, cancer. Any technique suitable for detecting gene copy number can be used in the practice of the present method, including Southern blotting and PCR amplification techniques. In a particular embodiment, the loss of a copy of the ARTS1 gene in an individual is inferred from loss of heterozygosity (LOH) at a chromosomal marker or gene that is closely linked to the ARTS1 gene. Methods for determining LOH of chromosomal markers are within the skill in the art.

In a further embodiment, the DNA methylation status of one or more regions of the ARTS1 gene in a sample from a subject is evaluated. An increase in the number of methylated nucleotide residues in an ARTS1 gene from a sample, relative to an ARTS1 gene from a control sample, also referred to herein as hypermethylation, is indicative of the subject having, or being predisposed to developing, cancer. As used herein, “DNA methylation status” refers to whether particular nucleotide residues in a given region of DNA are methylated or unmethylated. In mammalian cells, DNA methylation comprises addition of a methyl group to the 5-carbon position of cytosine (C) nucleotides to form 5-methylcytosine (5 mC) or methylcytosine. Only cytosines located 5′ to guanines (G) in CpG dinucleotides are methylated in mammalian cells. However, not all CpG dinucleotides are methylated. The pattern of methylation (i.e., whether a particular CpG dinucleotide is methylated or not) is relatively constant in cells (i.e., is maintained as cells divide), but can change under various circumstances. For example, methylation patterns in cells can change in certain human cancers.

Assays for detecting the methylation status of DNA are well known in the art (see, e.g., U.S. Pat. No. 6,858,388, to Markowitz, et al.). One type of assay for detecting methylated nucleotides is based on the treatment of genomic DNA with a chemical compound that converts unmethylated C, but not methylated C, to a different nucleotide base. In one embodiment of the invention, the DNA methylation status of a region of the ARTS1 gene is detected by a method that utilizes the compound sodium bisulfite. Sodium bisulfite converts C, but not 5 mC, to uracil (U). Methods for bisulfite treatment of DNA are known in the art (Herman, et al., 1996, Proc Natl Acad Sci U.S. Pat. No. 93:9821-6; Herman and Baylin, 1998, Current Protocols in Human Genetics, N. E. A. Dracopoli, ed., John Wiley & Sons, 2:10.6.1-10.6.10). When DNA that contains unmethylated C nucleotides is treated with sodium bisulfite, the sequence of that DNA is changed to yield a compound-converted DNA sequence. Detection of a U in the converted DNA sequence, therefore, is indicative of the presence of an unmethylated C in the original, unconverted molecule.

Methods for detecting converted bases in methylated DNA are well known in the art. One method of detecting U in compound-converted DNA sequences is called “methylation sensitive PCR” (MSP). In MSP, one set of primers, comprising a forward and a reverse primer, amplifies the compound-converted template sequence only if C bases in CpG dinucleotides within the ARTS1 DNA are methylated. These primers are called “methylation-specific primers.” Another set of primers amplifies the compound-converted template sequence only if C bases in CpG dinucleotides within the ARTS1 DNA are not methylated. This set of primers is called “unmethylation-specific primers.” Two separate PCR reactions, each using one set of the primers, are run simultaneously. In the case where C within CpG dinucleotides of the target sequence of the DNA are methylated, the methylation-specific primers, but not the unmethylation-specific primers, will amplify the compound-converted template sequence in the presence of a polymerase and a product will be produced. In the case where C within CpG dinucleotides of the target sequence of the DNA are unmethylated, the unmethylation-specific primers, but not the methylation-specific primers, will amplify the compound-converted template sequence in the presence of a polymerase and a product will be produced.

In an additional embodiment, the expression level of one or more ARTS1 gene products (e.g., RNA, protein) in a biological sample from a subject is determined. In one embodiment, a decrease in the level of expression of the gene product, relative to the expression level of the gene product in a control sample, is indicative of the subject having, or being at risk for developing, cancer.

In one embodiment, the ARTS1 gene product comprises RNA produced by transcription of all or part of the ARTS1 gene. Techniques for detecting the level of a given RNA transcript are well known in the art. These techniques include Northern blotting, RT-PCR and in situ hybridization, among others.

In the technique of Northern blotting, total cellular RNA can be purified from cells by homogenization in the presence of nucleic acid extraction buffer, followed by centrifugation. Nucleic acids are precipitated, and DNA is removed by treatment with DNase and precipitation. The RNA molecules are then separated by gel electrophoresis on agarose gels according to standard techniques, and transferred to nitrocellulose filters. The RNA is then immobilized on the filters by heating. Detection and quantification of specific RNA is accomplished using appropriately labeled DNA or RNA probes complementary to the RNA in question. See, e.g., Molecular Cloning: A Laboratory Manual, J. Sambrook, et al., eds., 2nd edition, Cold Spring Harbor Laboratory Press, 1989, Chapter 7, the entire disclosure of which is incorporated by reference. Suitable probes for Northern blot hybridization of an ARTS1 gene product can be produced from the nucleic acid sequences provided in FIG. 7 (SEQ ID NO. 1). Methods for preparation of labeled DNA and RNA probes, and the conditions for hybridization thereof to target nucleotide sequences, are described in Molecular Cloning: A Laboratory Manual, J. Sambrook, et al., eds., 2nd edition, Cold Spring Harbor Laboratory Press, 1989, Chapters 10 and 11, the disclosures of which are herein incorporated by reference.

The relative number of ARTS1 gene transcripts in cells from a biological sample can also be determined by reverse transcription of ARTS1 gene transcripts, followed by amplification of the reverse-transcribed transcripts by polymerase chain reaction (RT-PCR). The levels of ARTS1 gene transcripts can be quantified in comparison with an internal standard, for example, the level of mRNA from a “housekeeping” gene present in the same sample. A suitable “housekeeping” gene for use as an internal standard includes, e.g., myosin or glyceraldehyde-3-phosphate dehydrogenase (G3PDH). The methods for quantitative RT-PCR and variations thereof are within the skill in the art.

In another embodiment, the ARTS1 gene product is a protein encoded by the ARTS1 gene. Techniques for detecting protein levels are well known in the art and include, but are not limited to, Western blotting and immunohistochemical detection methods. A standard technique for assaying protein levels in a cellular extract is the Western blotting technique, described in Current Protocols in Molecular Biology, F. Ausubel, et al., eds., Vol. 2, John Wiley and Sons, Inc., 1998, Chapter 10, the entire disclosure of which is incorporated herein by reference. For example, cell extracts containing total cellular protein can be prepared by homogenizing cells in the presence of protein extraction buffer. The proteins are then separated by gel electrophoresis on polyacrylamide gels according to standard techniques, and transferred from the gel to nitrocellulose filters. Detection and quantification of a specific protein is accomplished using antibodies that recognize and bind to the protein of interest (e.g., antibodies that bind ARTS1).

The present invention also encompasses methods of preventing or treating a cancer caused by a defective ARTS1 gene. In one embodiment, the method involves administering an ARTS1 gene or gene product to a subject in need thereof (e.g., administering an ARTS1 protein, administering an ARTS1 nucleic acid (e.g., DNA, RNA)). In another embodiment, the method comprises replacing the defective ARTS1 -gene with a normal ARTS1 gene in the cells of the subject through “gene replacement” therapy, described previously herein.

As defined herein, a defective ARTS1 gene is any ARTS1 gene having an altered nucleotide sequence or structure that results in the reduction or ablation of one or more biological activities (e.g., tumor suppressor activity, pro-apoptotic activity) of an ARTS1 gene product. These defects include, but are not limited to, loss-of-function mutations, such as a nucleotide change(s) that results in the production of a mutant ARTS1 protein having no or partial biological activity, or a nucleotide change(s) that results in decreased expression of one or more ARTS1 gene products. In either case, any residual ARTS1 activity is insufficient to carry out the normal biological function of ARTS1. In a particular embodiment, the defect in the ARTS1 gene reduces or ablates the tumor suppressor activity of ARTS1.

Suitable cancers that can be treated or prevented by the methods of the invention include, but are not limited to, leukemia, melanoma, lymphoma, myeloma, pancreatic cancer, breast cancer, prostate cancer, colorectal cancer, lung cancer, ovarian cancer, kidney cancer, idiopathic pancytopenia, gastric cancer, Hodgkin's disease, non-Hodgkin's disease, esophogeal cancer, cervical cancer and thyroid cancer. In one embodiment, the cancer is selected from the group consisting of chronic lymphocytic leukemia (CLL), lung carcinoma, thyroid adenoma, kidney carcinoma, and essential thrombocytemia. In a particular embodiment, the cancer treated by the present method is chronic lymphocytic leukemia (CLL). In a further embodiment, the cancer treated by the present method is familial CLL. As used herein, familial CLL is defined as a type of CLL characterized by two or more cases of B-CLL in first-degree living relatives (N. Ishibe, et al. Leuk Lymphoma 42(1-2):99-108 (2001)). Other features of famillial CLL include, (i) 3 cases of CLL in 2 successive generations, (ii) the phenomenon of anticipation (i.e. earlier onset and more severe phenotype in successive generation) and (iii) a higher frequency of secondary tumors (R. S. Houlston, et al. Leuk. Res. 27:871-876 (2003)).

As used herein, the terms “treat”, “treatment” and “treating” refer to administration of one or more therapies (e.g., one or more therapeutic agents comprising the ARTS1 gene products (e.g., ARTS1 proteins and ARTS1 nucleic acids) of the invention) to reduce, ameliorate, or prevent the progression, severity and/or duration of a condition (e.g., cancer, tumor formation and growth), or to reduce, ameliorate, or prevent one or more symptoms (preferably, one or more discernible symptoms) of a condition (e.g., cancer). In specific embodiments, the terms “treat”, “treatment” and “treating” refer to the amelioration of at least one measurable physical parameter (e.g., tumor growth, metastasis) of a condition (e.g., cancer), not necessarily discernible by the patient. In other embodiments the terms “treat”, “treatment” and “treating” refer to the inhibition of the progression of a condition (e.g., cancer), either physically by, e.g., stabilization of a discernible symptom, physiologically by, e.g., stabilization of a physical parameter, or both. In other embodiments the terms “treat”, “treatment” and “treating” refer to the inhibition or reduction in the onset, development or progression of one or more symptoms associated with a condition.

As used herein, the terms “prevent”, “prevention” and “preventing” refer to the prophylactic administration of one or more therapies (e.g., one or more therapeutic agents comprising the ARTS1 gene products (e.g., ARTS1 proteins and ARTS1 nucleic acids) of the invention)) to reduce the risk of acquiring or developing a condition (e.g., cancer), or to reduce or inhibit the recurrence, onset or development of one or more symptoms of a particular condition (e.g., cancer). In a preferred embodiment, the ARTS1 protein of the invention is administered as a preventative measure to a patient, preferably a human, having a genetic or environmental risk factor for a condition (e.g., cancer).

As used herein, a “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

The ARTS1 gene products (e.g., ARTS1 proteins and ARTS1 nucleic acids) of the invention can be used as an in vivo or ex vivo therapeutic agent. For in vivo therapeutic use, the ARTS1 gene product (e.g., ARTS1 protein or ARTS1 nucleic acid) is administered, typically formulated with excipients and carriers as a pharmaceutical agent, to a patient in need thereof, e.g., one who is suffering from, or predisposed to, a cancer associated with a defect in the ARTS1 gene. The ARTS1 gene product (e.g., ARTS1 protein or ARTS1 nucleic acid) is formulated and administered to the patient in need thereof in an effective amount, i.e. an amount sufficient to effect the desired response, typically regression or inhibition of the tumor growth.

As used herein, an “effective amount” is the quantity of therapeutic agent in which a beneficial clinical outcome is achieved when the compound is administered to a subject. A “beneficial clinical outcome” includes therapeutic treatment of tumor cells, resulting in a reduction in the formation and growth of tumors. The amount of ARTS1 gene product (e.g., ARTS1 protein or ARTS1 nucleic acid) that will be effective in the prevention, treatment, management, and/or amelioration of a particular condition (e.g., cancer) or one or more symptoms thereof, will vary with the nature and severity of the disease or condition, and the route by which the gene product is administered. The frequency and dosage will also vary according to factors specific for each patient, e.g., the specific therapy administered, the severity of the disorder, disease, or condition (e.g., cancer), the route of administration, as well as age, body weight, response, and the past medical history of the patient. Effective doses may be extrapolated from dose-response curves derived from in vitro or animal model test systems. Suitable regiments can be selected by one skilled in the art by considering such factors and by following, for example, dosages reported in the literature and recommended in Hardman, et al., eds., 1996, Goodman & Gilman's The Pharmacological Basis Of Basis Of Therapeutics 9^{th} Ed, McGraw-Hill, New York; Physician's Desk Reference (PDR)57^{th} Ed., 2003, Medical Economics Co., Inc., Montvale, N.J., the entire teachings of which are incorporated herein by reference.

The exogenous sources of ARTS1 described herein (e.g., ARTS1 proteins, ARTS1 nucleic acids) can be administered to a subject by any conventional method of drug administration, for example, orally in capsules, suspensions or tablets, or by parenteral administration. Parenteral administration can include, for example, systemic administration, such as by intramuscular, intravenous, subcutaneous, or intraperitoneal injection. The compounds can also be administered orally (e.g., dietary), topically, by inhalation (e.g., intrabronchial, intranasal, oral inhalation or intranasal drops), rectally, vaginally, and the like. In a specific embodiment, local administration is a preferred mode of administration for treatment of cancers associated with ARTS1 gene defects (e.g., local administration site(s) of tumor formation).

The exogenous sources of ARTS1 described herein (e.g., ARTS1 proteins, ARTS1 nucleic acids) can be administered to the subject in conjunction with an acceptable pharmaceutical carrier or diluent as part of a pharmaceutical composition for treatment of a particular condition (e.g., a condition described herein). Formulation of the compound to be administered will vary according to the route of administration selected (e.g., solution, emulsion, capsule, and the like). Suitable pharmaceutically-acceptable carriers may contain inert ingredients which do not unduly inhibit the biological activity of the compounds. The pharmaceutically-acceptable carriers should be biocompatible, i.e., non-toxic, non-inflammatory, non-immunogenic and devoid of other undesired reactions upon administration to a subject. Standard pharmaceutical formulation techniques can be employed, such as those described in Remington's Pharmaceutical Sciences, ibid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents”, John Wiley and Sons, 1986).

### EXAMPLES

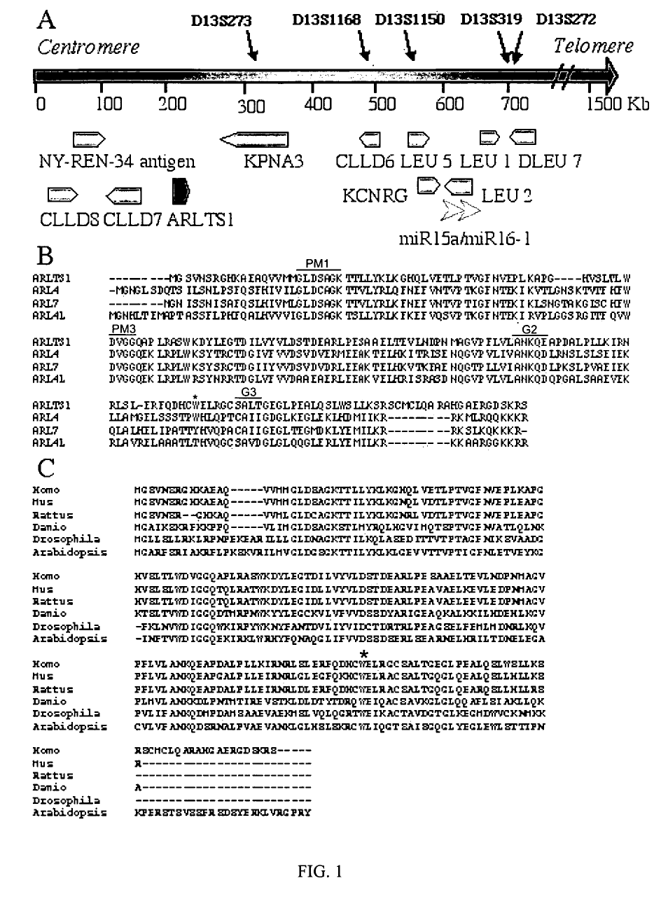

EXOFISH (Roest Crollius, et al., Nat Genet 25:235-8. (2000), which is incorporated herein by reference) was used to scan 1.4 Mb of assembled genomic sequence at chromosome 13q14 (Mabuchi, H., et al., Cancer Res 61, 2870-2877 (2001), Bullrich, F., et al., Cancer Res 61:6640-6648 (2001), Lander, E. S., et al., Nature 409:860-921 (2001), and Venter, J. C., et al., Science, 291:304-351 (2001), which are each incorporated herein by reference) for putative genes. A 182 bp ‘ecore’ (evoluntionary conserved region) coding for an aminoacidic sequence with high homology to several members of the ADP-ribosylation factor family was found. By using EST walking and RACE, the corresponding full-length cDNA was obtained. Comparison with the genomic sequence indicated that the cloned cDNA, which was designated ARTS1 (for ADP-Ribosylation factor-Like, putative Tumor Suppressor gene 1), derives from a small gene composed of two exons separated by a 1.8 kb intronic sequence and spanning about 6 kb of DNA. Using LOH analysis, it was found that this region was heterozygously deleted in a fraction of tumors, between 10% (colon cancers) and 20% (B-CLL). The putative ORF, within the second exon, encodes a 196-amino acid protein with a predicted molecular mass of 21 kDa. BLAST analysis and Conserved Domain search of protein databases revealed highly significant homology with the ADP-ribosylation factor (ARF) and ARF-like (ARL) protein subfamily of the ras family (Moss, J. & Vaughan, M. J Biol Chem 273:21431-21434 (1998) and Kahn, R. A., Der, C. J. & Bokoch, G. M. FASEB J 6:2512-2513 (1992), which are each incorporated herein by reference). At the protein level, related proteins share at most 45% identical amino acids. A multiple alignment with the CLUSTALW program indicates that ARTS1 belongs to the subgroup formed by ARL4, ARL6 and ARL7 (Jacobs, S., et al. FEBS Lett 456, 384-8. (1999)) (FIG. 1).

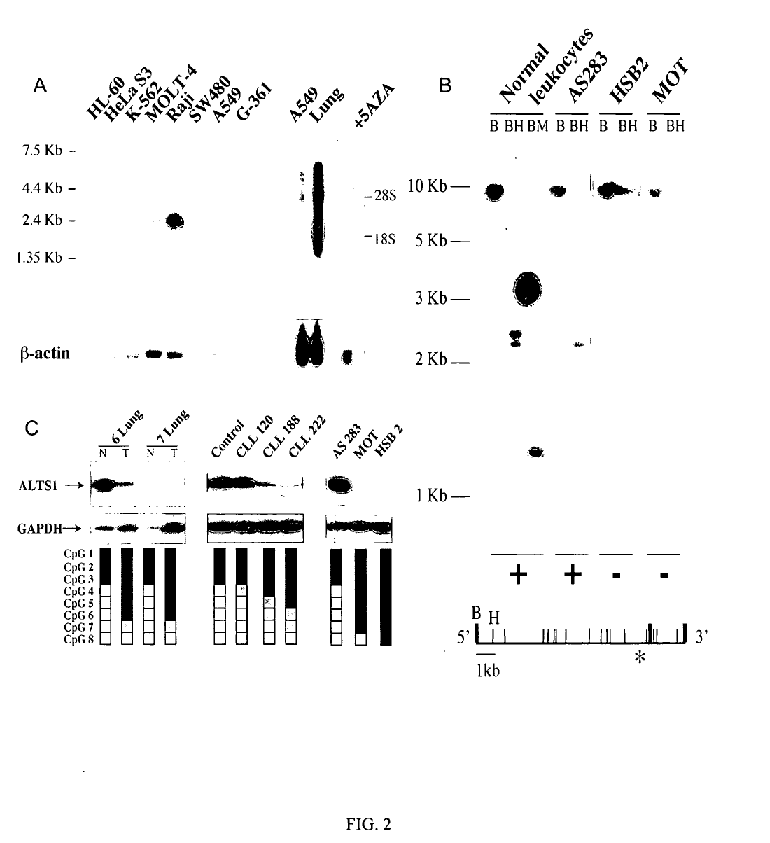

Northern analysis of normal human tissues with an ARTS1 probe revealed ubiquitous expression of a 2.2 kb transcript. In some tissues, two additional minor bands of approximately 1.3 and 5.5 kb were detected, resulting from the use of different polyadenylation sites (FIG. 4). The expression of ARTS1 was analyzed by Northern blot and/or semiquantitative RT-PCR in a set of 59 hematopoietic and solid tumor cell lines. ARTS1 expression was significantly reduced or absent in 22% (7/32) of blood cancer cell lines, 78% (7/9) of lung cancer cell lines, 33% (2/6) of esophageal cancer cell lines and 22% of pancreatic cancer cell lines, as well as in HeLa S3 (cervical carcinoma), SW 480 (colorectal cancer) and G-361 (melanoma) cell lines. In addition, 4 out of 16 fresh tumor samples (25%, 2/7 lung carcinomas and 2/9 B-CLL), for which cDNA and/or RNA were available, showed reduction or absence of ARTS1 expression when compared to their normal tissue counterparts (FIG. 2 and Table 5).

The possibility that, as occurs with other cancer-related genes such as TSLC1 (Kuramochi, M., et al. Nat Genet 27, 427-30. (2001) which is incorporated herein by reference) or p16 (Merlo, A., et al. Nat Med 1, 686-92. (1995) which is incorporated herein by reference), ARTS1 is downregulated through hypermethylation of the putative promoter, was examined. First, the global methylation level around ARTS1 was analyzed by Southern blotting using cell lines for which expression data was available. The level of expression is correlated with the methylation status of the genomic region—cell lines with low or no ARTS1 expression are highly methylated, while cell lines with normal levels of expression display only one methylated site (FIG. 2). ARTS1 DNA methylation patterns were examined in more detail through bisulfite sequencing to determine the methylation status of 5 CpG sites near the putative promoter sequence. Fresh tumor samples and tumor cell lines with low or absent ARTS1 expression showed higher methylation levels than normal tissues or tumors with normal expression levels (FIG. 2 and Table 5).

During an initial screening for mutations using 80 cell lines (including 70 used for gene expression and 10 melanoma cell lines), three mutations were identified in the ARTS1 open reading frame (ORF) (SEQ ID NO:15). The first, a missense mutation G446A (Trp149Stop), is present in homozygosity in the MCF7 breast cell line and in heterozygosity in the HS776T pancreatic carcinoma cell line. Two heterozygous substitutions were identified in melanoma cell lines: a T50C (Met17Thr) substitution and a C262A (Leu88Met) substitution in one of the patients with T50C (Table 1).

In order to establish the significance of these mutations, three panels of samples were screened (Methods and Table 6). The first included 216 human tumors that were screened by direct sequencing of the ARTS1 ORF (SEQ ID NO:15). Eight cases carried the G446A (Trp149Stop) mutation, including 3 breast cancers (3/48 of cases, 6.25%), 2 colorectal carcinomas (2/58, 3.45%), 1 lung carcinoma (1/5, 20%), 1 thyroid tumor (1/65, 1.5%) and one idiopathyc pancytopenia. All tumor samples had both the wild-type and mutant alleles, except for a breast tumor with LOH at the ARTS1 locus, which was homozygous for the mutation. Sequencing of the ARTS1 in paired normal tissues, which were available for three out of six tumors, revealed the same alteration in the germline of patients.

The second panel contains 109 blood DNAs, from patients with multiple cancers or with a family history of cancer, screened by direct sequencing. Six additional cases with the G446A (Trp149Stop) were identified−2 malignant melanomas+prostate carcinoma cases (2/17, 11.75%), 2 cases of familial CLL (2/17, 11.75%), 1 case of pancreatic cancer+melanoma (1/6, 16.5%) and 1 breast cancer case (1/69 of cases, 1.5%) (see Table 2 for family history). At the protein level, the stop codon inserts a premature termination 48 amino acids before the C-terminus, leading to the synthesis of a smaller protein with 148 instead of 196 amino acids (FIG. 1). Thus, the truncated protein lacks the C-terminal motif presumably involved in nucleotide binding and hydrolysis characteristic of Ras related GTPases, one of the five additional amino acids typical of the ARF subfamily (Gly161) and the putative nuclear localization signal. Furthermore, Trp149, the site of the mutation is conserved in ARL4 and in 11 other ARF or ARF related genes, including all six ARF genes.

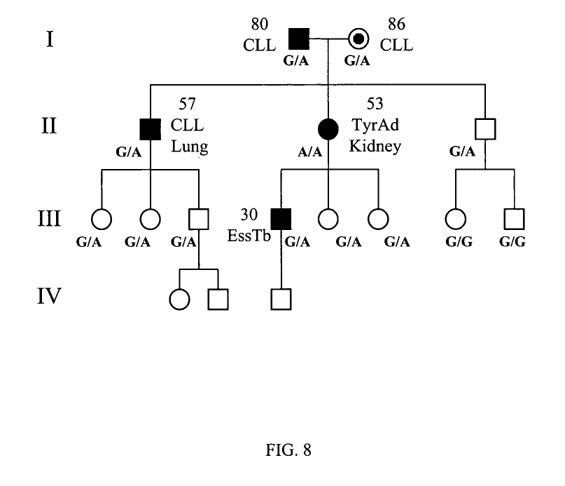

In one kindred with familial CLL, all five family members who have the cancer harbored the G446A (Trp 149Stop) polymorphism, whereas the two unaffected family members did not (FIG. 8). The only member of this kindred with a homozygous mutation developed kidney carcinoma and thyroid adenoma when he was less than 50 years old. In the third generation, there are six members who harbor the polymorphism, including one diagnosed with Essential Thrombocytemia (a premalignant state). Potential for cancer development in the other five individuals from this generation who have the polymorphism could not be assessed, as each of these individuals is less than 40 years old.

The third panel comprises the case-controls: allele frequency for the G446A (Trp149Stop) mutation in three separate Caucasian cohorts was 2.10%, with variations between 0.86% (1/116) in the U.S. population and 3.44% (7/203) in the Italian population. Overall, 14 patients out of 325 analyzed (4.63%) and 10 out of 475 normal controls (2.1%) had the stop mutation. The odds of G446A (Trp149Stop) were 2.10 (95% CI 0.92-4.77) times higher in cancer patients versus controls. After stratification upon family history of cancer, these odds increase in the group with positive family history to 2.70 (95% CI 0.85-8.32) (Table 6). In addition to the G446A (Trp149Stop), several other variants in the ARTS1 gene were identified, including a G490A (Glu164Lys) substitution in a thyroid adenoma (Table 1). Four mutations in a total of 64 analyzed thyroid adenomas and carcinomas were found (two C65T missense mutations, one G446A nonsense mutation and one G490A missense mutation). All four mutations were found in adenomas of follicular origin, whereas all samples of non-follicular histotype (42/65, 65%) were wild-type. It is highly unlikely that this allelic distribution is random (P=0.01 at Fisher exact test). Also, a G446A homozygous patient in a family with CLL has thyroid adenoma (Table 2). Taken together, these observations raise the possibility that ARTS1 is involved in a portion of thyroid tumors with follicular histotype.

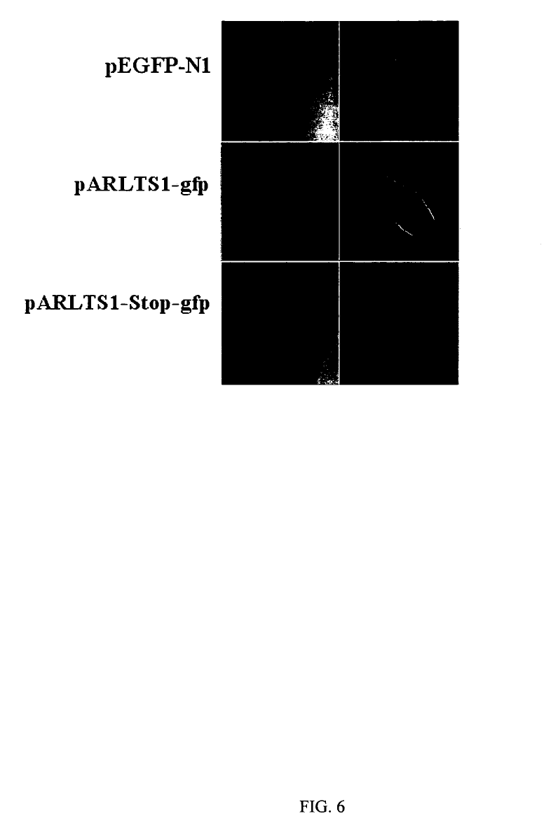

ARTS1 appears to be the first ARF family member reported to be altered in human cancers. Because of their nuclear localization signal (NLS), ARL4, ARL6 and ARL7 appear to be cargo molecules transported via the translocators importin-á and âinto the nucleus, where they have yet unknown functions. Of note, ARTS1 lacks a classical NLS at its C-terminus, and probably contains an a typical NLS. Using GFP constructs, the wild-type ARTS1 protein (PARLTS1-gfp) was shown to be localized both in the nucleus and in the cytoplasm. The mutant ARTS1 ÄC-terminus protein (PARLTS1-Stop-gfp) has the same intracellular protein (FIG. 6). ARTS1 may be involved in novel cytoplasmic/nuclear membrane trafficking and/or signaling cascades that are important in different types of cells.

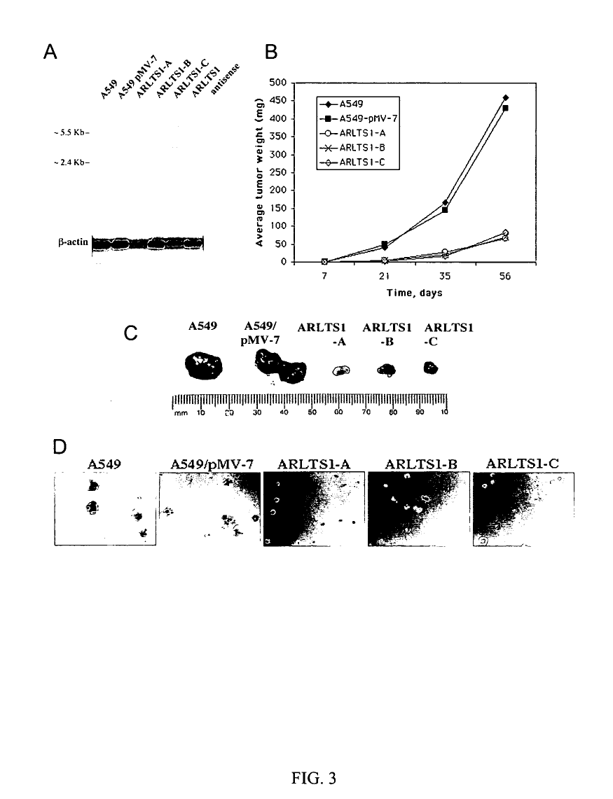

Northern and RT-PCR expression data showed that ARTS1 expression was dramatically decreased in A549, a highly tumorigenic non-small cell lung carcinoma (NSCLC) cell line (Fogh, J., Fogh, J. M. & Orfeo, T. J Natl Cancer Inst 59, 221-6 (1977), which is incorporated herein by reference), when compared to the level found in normal lung. The ARTS1 ORF, under the control of the LTR promoter, was transfected into A549. Several stable clones were obtained and five of them were used in experiments: parental A549, the A549-pMV-7 (empty vector) clone, and three neomycin-resistant transfectants (ARLTS1-A, ARLTS1-B, and ARLTS1-C) selected according to the level of expression of the transfected ARTS1 minigene (FIG. 3). To evaluate the biological effect of ARTS1 in vitro and in vivo, tumorigenicity was examined by soft agar and in Nu/Nu nude mice (FIG. 3), which lack an immune system. All three transfected clones give rise to smaller colonies with a shorter survival in comparison to the parental cell or cells transfected with the empty vector. Furthermore, during 10 weeks of observation after the subcutaneous injection, the former consistently formed smaller, nonprogressive tumors, while the latter formed large, progressively growing tumors in nude mice. Thus, ARTS1 by itself has significant tumor-suppressor activity in A549 cells.

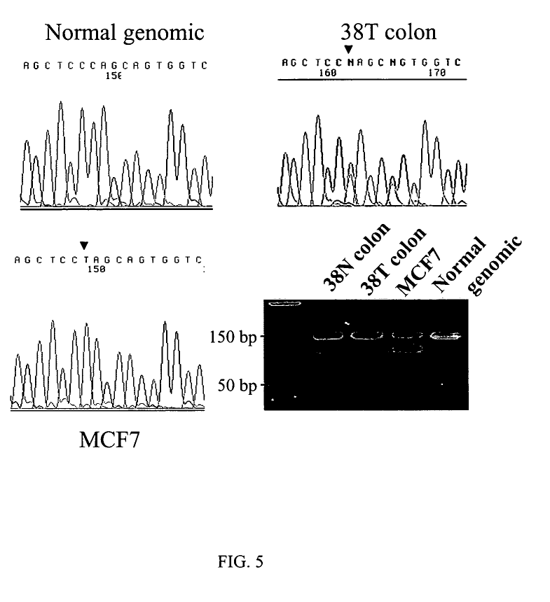

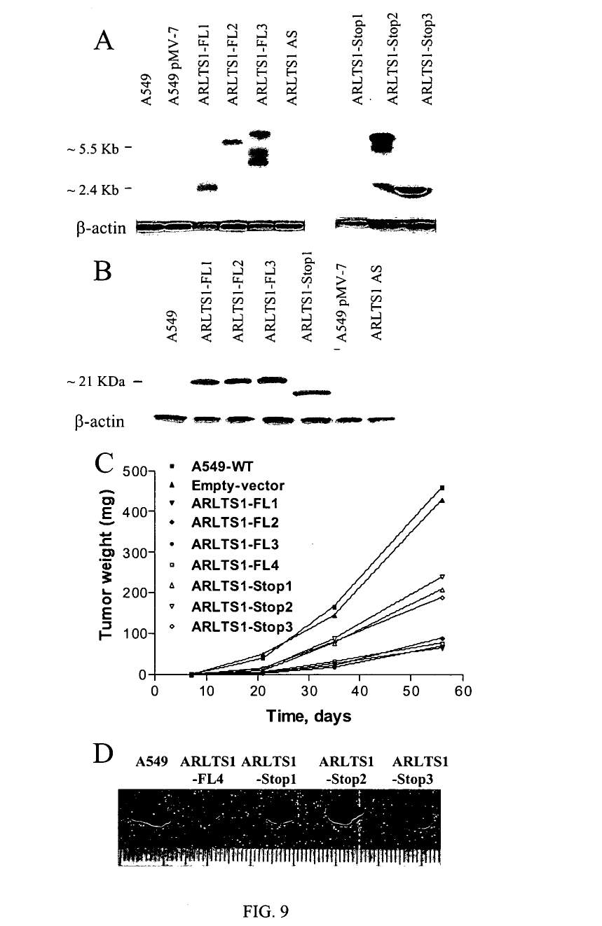

A549 cells were also transfected with a pMV-7 vector containing an ARTS1 cDNA harboring the G446A polymorphism, denoted as ARLTS1-Stop1, ARLTS-Stop2 and ARLTS-Stop3 in FIG. 9). These transfected cells, which express a truncated ARTS1 protein (FIG. 9B), were tested for the ability to form tumors in Nu/Nu mice. During 8 weeks of observation, tumor size was found to be intermediate in the group of mice injected with A549 clones expressing the truncated ARTS1 protein (50% weight reduction), when compared to tumor size in mice injected with either clones that expressed full-length ARTS1 (ARLTS1-FL1, ARLTS1-FL2, and ARLTS1-FL3) or clones of untransfected A549 cells (A549) (FIGS. 5 and 9C-D). The difference between the size of tumors in mice expressing ARTS1-FL and ARTS1-Stop was statistically significant (P=0.04). Thus, ARTS1 displays tumor-suppressor activity in A549 cells, while the truncated ARTS1 protein displays only partial activity, indicating that the G446A polymorphism has functional implications.

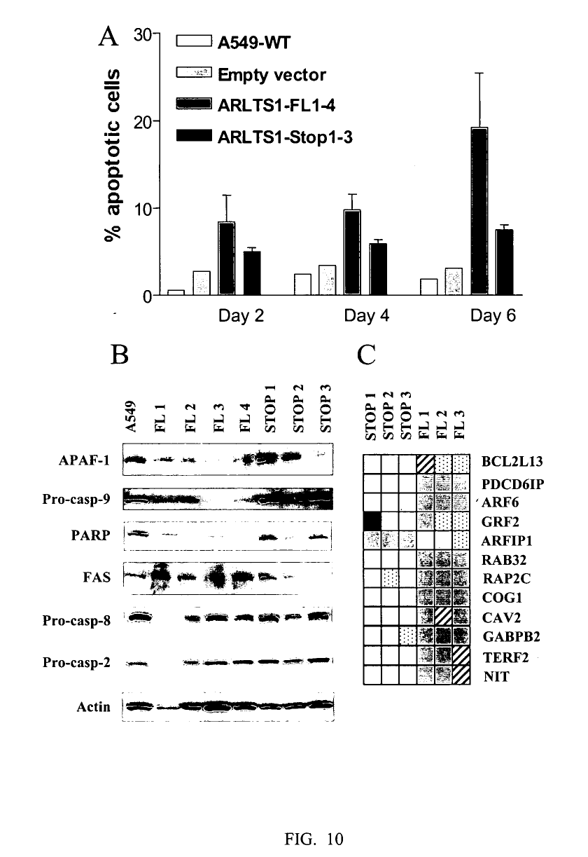

We found that a higher percentage of cells transfected with full length ARTS1 (ARLTS-1-FL) undergo apoptosis as compared to the parental cells, while the G0/G1 and S phase populations did not differ significantly. By contrast, induction of apoptosis in cells expressing the truncated protein was less effective than in cells expressing the full-length protein (P=0.007) (FIG. 10A). Western blots (FIG. 10B) showed different levels of “apoptosome” complex molecules APAF-1 and pro-caspase-9, and the effector protein PARP in full-length ARTS1-transfected cells (FL 1, FL 2, FL 3 and FL 4), as compared to cells transfected with the truncated ARTS1 gene (STOP 1, STOP 2, STOP 3), with higher levels of activation in the former data in accordance with the Caspase 3 assay.

A549 cells transfected with full-length ARTS1 minigenes have a different gene expression profile than A549 cells transfected with ARTS1 minigenes harboring the G446A polymorphism (FIG. 10C). The “Stop” transfectants had lower levels of “pro-apoptosis” transcripts (such as BCL2L13, P=0.003) when compared with full-length ARTS1-expressing clones (e.g., FL1, FL2, FL3, and FL4). This difference was statistically significant. Furthermore, several members of the small GTPases family (for example, ARF6, P=0.005) were expressed at statistically lower levels in the “Stop” transfectants. Collectively, the data presented in FIG. 10 suggest that the full-length ARTS1 protein confers a greater propensity for cells to undergo apoptosis.

The presence of a new tumor suppressor gene within the well-characterized superfamily of Ras oncogenes is not contradictory. It was recently shown that wild-type Kras2 could inhibit lung carcinogenesis in mice, clearly illustrating the tumor suppressor role of the gene in lung tumorigenesis (Zhang, Z., et al., Nat. Genet 29: 25-33. (2001), which is incorporated herein by reference). The principal mechanism for ARTS1 inactivation in human cancers is biallelic methylation, as was proposed in the revised Knudson's two hit hypotheses (Jones, P. A. & Laird, P. W., Nat. Genet. 21: 163-7. (1999), which is incorporated herein by reference). One intriguing aspect of ARTS1 involvement in human cancer is the real significance of the G446A (Trp149Stop) nonsense mutation. Because the frequency of G446A mutation is about three times higher in familial cancers as in the general population and about two times higher as in sporadic cancers, one possible explanation is that ARTS1 germline mutations have low penetrance and are associated with a small percentage of familial melanoma or familial CLL cancers (which harbor a ten times higher frequency of the truncating mutation as in the same population control group). According to this, it is possible that there exists kindreds which carry the mutation but do not develop cancer. The same is also true for some other TSGs, as is the case of BRCA2 germline mutations in breast and pancreatic cancers (Goggins, M., et al., Cancer Res 56, 5360-4. (1996), which is incorporated herein by reference). An alternative explanation is that this truncating mutation does not have a pathogenetic role in human cancers, because the lost domains are not important for tumorigenesis or because the protein has redundant functions with other ARL family members. Until now, only one polymorphic stop codon was identified in cancer related genes, the Lys3326ter in BRCA2 gene (Mazoyer, S., et al., Nat Genet 14, 253-4. (1996), which is incorporated herein by reference). However, until independent groups analyze a larger number of cases, the possibility that such polymorphisms are associated with a modest increased cancer risk, or are associated with other phenotypes in the heterozygous or homozygous state, cannot be excluded.

Methods

Cell Lines. Eighty cell lines derived from human tumors were used in this study. Forty-four were hematopoietic cancer cell lines and 36 were solid tumors cell lines (for detailed list, see Table 3). As controls, six lymphoblastoid cell lines, made from peripheral blood lymphocytes of patients with Alzheimer's disease by transformation with Epstein Barr Virus (EBV), were used. All the cell lines were obtained from the American Type Culture Collection (ATCC) (Manassas, Va.) and maintained according to ATCC instructions.

Patient Samples. Experimental samples were derived from sporadic tumors or from peripheral blood of patients with familial cancer (total of 325). Control samples were derived from the blood of patients with diseases other than cancer or from healthy individuals (total of 475). All samples were obtained with informed consent following institutional guidelines for the protection of human subjects. The 216 human sporadic tumors analyzed included 65 thyroid tumors, 58 colorectal adenocarcinomas, 48 breast carcinomas, 39 B-CLLs, 5 lung carcinomas and 1 idiopathyc pancytopenia. The panels of DNA from blood include: a) 69 DNA samples from females with BRCA-1-negative/BRCA-2-negative familial breast cancer; b) 17 DNA samples from males affected with prostate cancer and malignant melanoma, which had been found negative for mutations at the p16 locus; c) 17 DNAs from patients with familial CLL (at least two first-degree relatives affected); and d) 6 DNAs from individuals with pancreatic cancer or melanoma who have a family history of at least one case of melanoma or pancreatic cancer and who are negative for mutations in the p16 and p14 genes. Patients' profiles were similar for both groups: about 60% of cancer patients were from European Caucasian origin and the remaining 40% were from U.S. persons. In the control group, the proportions of the two cohorts were 75% and 25%, respectively. No bias toward distinct population groups (such as Ashkenazim) was noted. High molecular weight (HMW) DNA was extracted by conventional protocols (Sambrook, J., Frisch, E. F. & Maniatis, T. Molecular cloning: A Laboratory Manual, (Cold Spring Harbor Laboratory Press, Cold Spring Harbor, N.Y., 1989), which is incorporated herein by reference).

Rapid Amplification of cDNA Ends (RACE). The 3′ and 5′ ends of mRNAs were obtained by RACE from human testis, fetal liver, bone marrow and lymph node, using the Marathon-ready and the SMART RACE protocols (Clontech, Palo Alto, Calif.). The PCR products were separated on 1.0-2.0% agarose gels and gel purified using the QIAquick gel extraction kit (QIAGEN) or cloned into the TA vector using TOPO TA Cloning (Invitrogen Carlsbad, Calif.) and sequenced.

Northern blot analysis. Multiple human tissue Northern blots were purchased from Clontech and total RNA was extracted from tumor cell lines or tumors by the QIAGEN RNeasy mini kit (QIAGEN) according to the manufacturer's protocol. The membranes were hybridized with a 443-bp probe containing the majority of the ARTS1 open reading frame (ORF), labeled with ³²P dCTP by random priming (Prime-it II Kit, Stratagene). Prehybridization and hybridization were carried out in Church Buffer (7% SDS, 0.5M phosphate buffer pH 7.2, 10 mM EDTA) for 18-20 h at 65° C. as described in Sambrook (supra).

Reversed Transcription PCR (RT-PCR) analysis. The DNA sequence was confirmed by RT-PCR and a semiquantitative RT-PCR was performed to analyze the levels of gene expression in various normal and tumor tissues. Five microliters of cDNA were used for each PCR with Advantage2 PCR kit (Clontech) and 10 pmol of each gene-specific primer for 35 cycles of 94° C. for 20 s, 65° C. for 30 s, 68° C. for 1 min (for a complete list of primers used in this study, see Table 4). To ensure that the RNA was of sufficient purity for RT-PCR, a PCR assay with primers specific for GAPDH cDNA (Clontech) was used. Semiquantitative PCR was performed with 23 cycles of amplification for ARTS1 and 18 cycles for GAPDH, in order to remain within a range of linear increase in the amount of PCR product. RT-PCR products were separated by agarose gel electrophoresis and blotted on Hybond N+nylon membranes following standard procedures in Sambrook (supra). Membranes were hybridized with the same probe and under the same conditions as for Northern blotting. The relative intensity of hybridization signals was analyzed with a Phospholmager system (Molecular Dynamics).

Methylation analysis by Southern blotting. In order to identify the global level of methylation for the ARTS1 locus, five micrograms of total genomic DNA were digested with Bg1II alone or in combination with methylation-sensitive HpaII (Roche) using a total of 40 U of enzyme for 12 h. Digests were electrophoresed on 0.8% agarose gels and blotted on Hybond N+positively charged nylon membranes (Amersham Pharmacia Biotech) and hybridized with the same ORF probe as previously described.

Methylation-sensitive PCR. To analyze methylation levels in the 5′ upstream region of ARTS1, a region upstream of the first exon of ARTS1 was amplified and bisulfite sequencing was carried out as described in Frommer, M., et. al., Proc. Natl. Acad. Sci. USA 89:1827-1831, (1992), which is incorporated herein by reference. Modified DNA (200 ng) was subjected to PCR. PCR products were purified and directly sequenced in order to obtain average methylation levels. In addition, PCR products were subcloned and at least six clones were sequenced to confirm direct sequencing data. Because of the unavoidable contamination of normal cells in the tumor specimens, we defined a CpG site as “hypermethylated” when more than 70% of PCR products contained bisulfite-resistant cytosines; “partial methylation” indicates detection of these products in 20-70% of the total products.

LOH studies. The paired normal and colorectal tumor DNA samples were tested for LOH by PCR amplification with oligonucleotide primers for microsatellite markers at D13S165 and D13S273 using fluorescent-labeled primers (ABI). One single nucleotide polymorphism found inside the ORF of ARTS1 (T442C) was heterozygous in about 45% of sequenced samples and was very useful for the rapid discrimination of informative/noninformative patients. The amplification products were run on an Applied Biosystems Model 377 DNA sequencing system (PE, Applied Biosystems). The LOH data for 39 paired normal/tumor B-CLL samples used in this study were previously reported in Bullrich, F., et al., Blood 88, 3109-15 (1996), which is incorporated herein by reference.

Mutation detection. Primers used in mutation analysis were designed from intronic sequences directly upstream of the second exon and within the 3′ UTR region of ARTS1. PCRs were carried out for 35 cycles of 94° C. for 30 s, 62° C. for 30 s and 72° C. for 1 min using RedTaq genomic DNA polymerase (Sigma-Aldrich, St. Louis, Mo.), purified with the QIAquick PCR purification kit (QIAGEN) and then both strands were directly sequenced using the Applied Biosystems Model 377 DNA sequencing system (PE, Applied Biosystems, Foster City, Calif.). The 203 normal controls from the Italian population were analyzed by denatured high-performance liquid chromatography (DHPLC) (Transgenomics, Omaha Nebr.). The temperature used for heteroduplex formation was 57° C. and all the samples with abnormal patterns were directly sequenced.

Subcellular localization. The pEGFP N1-ARTS1 vector was prepared by digesting pEGFP N1 (Clontech) with SmaI; the insert was obtained by amplifying the ARTS1 full-length insert with Pfu where its stop codon was eliminated in order to generate an ARTS1-EGFP protein fused at the C-terminus. An additional pEGFP N1-ARTS1 ÄC-terminus vector was prepared carrying an ARTS1 gene harboring the G446A mutation. 293 cells were transfected by calcium phosphate (ProFection from Promega, Madison Wis.) and cultured on a cover slip. 24-48 h after transfection, cells were analyzed by fluorescence microscopy, as described in Ghosh, K. & Ghosh, H. P. Biochem Cell Biol 77, 165-78 (1999), which is incorporated herein by reference.

Stable transfection of A549 cells. A549 cells were cultured in RPMI supplemented with 10% fetal bovine serum. ARTS1 expression vectors, p-MV7-ARTS1-sense and p-MV7-ARTS1-ΔC-terminus, were constructed by ligating the open reading frame of either ARTS1 or the G446A ARTS1 polymorphic variant in a sense orientation into the mammalian expression vector, pMV-7. All constructs were sequenced in order to exclude random mutants and were transfected by FuGENE6 transfection reagent according to the manufacturer's protocol (Boehringer Mannhiem). Transfected cells were selected with G418.

Analysis of transformed phenotype. Soft-agar colony assays of A549 wild-type, Transfectants carrying full length ARTS1 (ARTS1-FL) and transfectants carrying the truncated ARTS1 gene (ARTS1-Stop) were performed as described in Trapasso, F., et al. Mol Cell Biol 20, 9236-46 (2000), which is incorporated herein by reference. A suspension of 106 cells in PBS (0.2 ml) was injected subcutaneousy into the right flank of Nu/Nu athymic mice (Jackson Laboratories Charles River, Cambridge, Mass.). Mice were sacrificed after 1, 3, 5, and 8 weeks and tumors were removed, weighed and measured in three dimesions to assess the effects of ARTS1-FL or ARTS1-Stop proteins on tumorigenesis, in vivo. All experiments were performed in accordance with institutional guidelines.

Cell cycle profiles were generated using flow cytometry on propidium iodide stained cells and apoptosis was monitored using the Active Caspase-3 PE Mab Apoptosis kit (PharMingen, BD Biosciences, Mohrsville, Pa.). Gene expression profiles were generated using a KCC/TJU human 18.5K Expression Bioarray (Compugen Human Oligo Set 1.0). Gene expression profiling was performed as described (Ramakrishnan R., et al., Nucleic Acids Res. 30:e30 (2002)).

Statistical analysis. Statistical analysis of results was performed using the Fisher's exact test; a P value of <0.05 was considered statistically significant. The cancer risk associated with the specific mutations identified in this study was analyzed using the odds ratio (OR) from a logistic regression model. Tumor weights in immunodeficient mice were examined in an analysis of variance model, which included a treatment group and the time at which the animal was sacrificed. Two-sided p-values for specific contrasts between groups are provided. P values <0.05 were considered statistically significant.

TABLE 1
ARTS1 sequence analysis in human cell lines, tumors and normal controls.

| | Amino | Amino acid | Cell | Sporadic | Familial | Normals, |
|---|---|---|---|---|---|---|
| Variant | acid | conservation | Lines | tumors | cancers, | blood |
| name¹ | Change | (%)² | (%) | (%) | blood (%) | (%) |
| T50 to C | Met 17 | 9/14 (65) | 2/80 (2.5)³ | 0/216 | 0/109 | 0/272 |
| | to Thr | | | | | |
| C65 to T | Ser 22 | 3/14 (21), PM1 | 0/80 | 2/216 (1)⁴ | 0/109 | 1/272 (0.4) |
| | to Leu | site | | | | |
| C262 to A | Leu 88 | Leu only in | 1/80 (1)³ | 0/216 | 0/109 | 0/272 |
| | to Met | ARTS1 | | | | |
| C392 to T | Pro | 4/14 (29) | 6/80 (7.5) | 14/216 (6.5) | 4/109 (4) | 17/272 (6.25) |
| | 131 to | | | | | |
| | Leu | | | | | |
| T442 to C | Cys | Cys only in | 25/80 (31) | 127/216 (59) | 80/109 (73) | 182/272 (67) |
| | 148 to | ARTS1 | | | | |
| | Arg | | | | | |
| G446 to A | Trp | 12/14 (86) | 2/80 (2.5) | 8/216 (3.7) | 6/109 (6) | 10/475 (2.1) |
| | 149 to | | | | | |
| | Stop | | | | | |
| G490 to A | Glu | 9/14 (65) | 0/80 | 1/216 (0.5)⁴ | 0/109 | 0/272 |
| | 164 to | | | | | |
| | Lys | | | | | |

Notes:
¹We identified also several synonymous polymorphisms such as: C175 to T (Leu 59); G 297 to A (Ser 99); C345 to T (Val 115); G396 to C (Leu 132); G546 to A (Gln 182).
²Data obtained by a multiple alignment of ARTS1 protein with ARF1 to ARF6 and ARL1 to ARL7 at the GenomeNet CLUSTALW server.
³Found only in melanoma cell lines.
⁴Found only in thyroid adenomas.

TABLE 2
Clinical data from families with G446A (Trp149Stop) mutation.

| Proband, sex, age¹ | Cancer type | Cancer Family history |
|---|---|---|
| KRR0003, female, 46 | B-CLL | Twin sister G446A +ve with B-CLL |
| TOR-1B, male, 57 | B-CLL and lung | Sister, 53, homozygous G446A with Thyroid |
| | cancer | adenoma; his son, 30, obligate carrier, |
| | | Essential thrombocytemia |
| | | Brother, heterozygous G446A - normal |
| | | Mother, dead, obligate carrier, B-CLL at |
| | | 80 yrs-old |
| | | Father, 86, obligate carrier, B-CLL |
| P/M 35003, male, ? | Gastric, 72 | None |
| | Melanoma, 72 | |
| | Prostate, 73 | |
| P/M 35012, male, ? | Prostate, 66 | Mother, cancer, unknown location, ? |
| | Melanoma, 67 | Brother, prostate, 73 |
| | | Sister, “black moles”, ? |
| | | Daughter, breast, ? |
| 1054-22671, male, | Melanoma, 50 | Paternal uncle, melanoma, ? |
| dead | Lung metastasis, 55 | Paternal aunt, pancreatic, ? |
| | | Paternal cousin, pancreatic, ? |
| | | Paternal cousin, head and neck, ? |
| 15-265-S87, female, ? | Bilateral breast | Daughter, 48, G446A carrier, unaffected |
| | cancer, 32 and 35 | |
| | Ovarian cancer, 50 | |

Note:
¹? = Age data unknown

TABLE 3
Cell lines used in the described experiments

| Hystotype | Cell lines used |
|---|---|
| Burkitt's lymphoma | AG876, AS283, BL2, BL30, BL41, CA46, |
| | DA978, Daudi, EB-B, ED36, Jiyoye, |
| | Lauckes, Nanalwa, P3HR-1, Raji, Ramos, |
| | RS11864, SKDHL and WMN |
| Multiple myeloma | HuNS1, MC/CAR, NC1-H929, RPM18226 |
| | and U266B1 |
| Large cell lymphoma | DB and SR |
| Immunoblastic B | JM1 |
| cell lymphoma | |
| Diffuse mix lymphoma | HT |
| Hodgkin's disease | RPM16666 and Hs445 |
| Non-Hodgkin's disease | RL |
| B-ALL | MV4; 11, RS4; 11, 697 |
| T-cell lymphomas | CEM, Del 1, HH, HSB2, HuT 102, MOLT-3, |
| and leukemias | MOLT-4, and MJ |
| Hairy cell leukemia | Mo T |
| CML-Erythroid leukemia | K562 |
| Lung carcinomas | A549, AFL, Calu-3, H69, H460, H1299, |
| | SKMES, 498 and 1285 |
| Pancreatic carcinomas | AsPC1, BxPC3, Capan-2, CFPAC-1, HS766T, |
| | MiaPaca, PANC1, PSN1, and SU8686 |
| Esophageal cancers | TE1, TE2, E10, TE15, KY200 and KY300 |
| Malignant melanoma | M14, 1007 MP, IR 6, WM 266.4, 397 MEL, |
| | 13443 and four cell lines derived from |
| | melanoma patients |
| Colon carcinoma | LoVo |
| Cervical carcinoma | HeLa |

TABLE 4
Primers used in the described experiments

| Primer | | |
|---|---|---|
| name | Primer sequence (5′-3′) | Application |
| 3′-ex2F | 5′-CCA TGG GTT CTG TGA ATT | Northern blot |
| | CCA GAG G (SEQ NO:5) | analysis |
| 5′-ex2R2 | 5′-CAG TGG TCC TGG AAT CTC | |
| | TCT AGA C (SEQ ID NO:6) | |
| 3′ex1F | 5′-GCC AGC AGA AAG CAG CTC | Reversed |
| | CAT AGG (SEQ ID NO:7) | Transcription |
| | | PCT (RT-PCR) |
| | | analysis |
| 5′ex2R1 | 5′-TTC AGG AGG CTC CAC AGG | |
| | CTC TGC (SEQ ID NO:8) | |
| MET-F | 5′-GAG GTA TGT ATT GAA AG | Methylation- |
| | AAG AGG (SEQ ID NO:9) | specific PCR |
| MET-R | 5′-AAC AAA ACC CAA TAA CAA | |
| | CTC CA (SEQ ID NO:10) | |
| ORF-F1 | 5′-CAG AAG ACA GTA GCT GAT | Genomic Mutation |
| | GTG (SEQ ID NO:11) | detection |
| ORF-R2 | 5′-GAG CAA AGA TAT GCT GCT | |
| | CTG (SEQ ID NO:12) | |
| MaeI-F1 | 5′-GCT GAG TCC AGA GAG ATT | G446A |
| | CCA GG (SEQ ID NO: 13) | (Trp149Stop) |
| MaeI-R1 | 5′-TCT CGC CTG CAG ACA CAT | detection by |
| | GC (SEQ ID NO:14) | MaeI digestion |

TABLE 5
Expression levels and methylation status of the ARTS1
promoter in human cancer cell lines

| | | ARTS1 | |
|---|---|---|---|
| Name | Origin | expression^{a} | Methylation |
| Normal lung 1 | Normal lung | + | Low |
| Normal lung 2 | Normal lung | + | Low |
| A 549 | Lung carcinoma | . | Hypermethylation |
| AFL | Lung carcinoma | ./+ | Hypermethylation |
| Calu-3 | Lung carcinoma | ./+ | ND |
| H 1299 | Lung carcinoma | . | Hypermethylation |
| H 69 | Lung carcinoma | ./+ | ND |
| 1285 | Lung carcinoma | ./+ | ND |
| H 460 | Lung carcinoma | ./+ | Hypermethylation |
| Lymphoblastoid | Immortalized | + | Low |
| 1 | lymphoblasts | | |
| Lymphoblastoid | Immortalized | + | Low |
| 2 | lymphoblasts | | |
| Del 1 | T cell lymphoma | ./+ | Hypermethylation |
| HH | T cell lymphoma | . | Hypermethylation |
| HSB 2 | T cell ALL | . | Hypermethylation |
| HuT 102 | T cell lymphoma | . | Hypermethylation |
| K 562 | CML-Erythroid | . | ND |
| | leukemia | | |
| MJ | T cell lymphoma | . | Hypermethylation |
| Mo T | T cell lymphoma | . | Hypermethylation |
| AS 283 | Burkitt's lymphoma | + | Low |
| BL 41 | Burkitt's lymphoma | + | Low |
| PSN 1 | Pancreatic | ./+ | Hypermethylation |
| | carcinoma | | |
| MiaPaca | Pancreatic | ./+ | Hypermethylation |
| | carcinoma | | |
| HeLa | Cervical carcinoma | . | Hypermethylation |
| SW 480 | Colon carcinoma | . | ND |
| G-361 | Melanoma | . | ND |

^{a}+, normal expression; +/−, reduced expression; and −, absent expression;
ND—not done

TABLE 6
Allele frequency of G446A (Trp149Stop) in unrelated cancer patients and control cases.

| Cancer patients | | | | Normal controls | | |
|---|---|---|---|---|---|---|
| | | Sample | | | Sample | |
| | | size, | | | size, | |
| Tumor type | Source | origin | G446A | Source | origin | G446A |
| Colorectal cancers | Bucharest, | 58, tumor | 2 | Philadelphia | 116, blood | 1 |
| “sporadic” | Romania | | | | | |
| Breast “sporadic” | Ferrara, | 38, tumor | 3 | Bucharest | 156, blood | 2 |
| | Italy | | | | | |
| Breast “sporadic” | Aarhus, | 10, tumor | 0 | Ferrara | 203, blood | 7 |
| | Denmark | | | | | |
| CLL “sporadic” | US | 39, tumor | 0 | | | |
| Lung “sporadic” | Milan, | 5, tumor | 1 | | | |
| | Italy | | | | | |
| Thyroid “sporadic” | Catanzaro, | 65, tumor | 1 | | | |
| | Italy | | | | | |
| CLL familial | Paris, | 11, blood | 1 | | | |
| | France | | | | | |
| CLL familial | US | 6, blood | 1 | | | |
| Breat familial | Philadelphia, | 69, blood | 1 | | | |
| | PA | | | | | |
| Melanoma + prostate | Philadelphia, | 17, blood | 2 | | | |
| | PA | | | | | |
| Pancreatic + melanoma | Philadelphia, | 6, blood | 1 | | | |
| | PA | | | | | |
| Idiopathyc | Bucharest, | 1, blood | 1 | | | |
| Pancytopenia | Romania | | | | | |
| Total | | 325 | 14 | | 475 | 10 |
| | | | (4.30%) | | | (2.10%) |

The relevant teachings of all publications cited herein that have not explicitly been incorporated by reference, are incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

1. An isolated protein comprising the amino acid sequence of SEQ ID NO:2.

2. An isolated nucleic acid molecule that comprises a nucleic acid sequence that encodes the protein of claim 1.

3. An isolated nucleic acid molecule comprising SEQ ID NO:1 or a fragment thereof having at least 10 nucleotides.

4. The nucleic acid molecule of claim 3 consisting of SEQ ID NO:1.

5. A recombinant expression vector comprising the nucleic acid molecule of claim 3.

6. A host cell comprising the recombinant expression vector of claim 5.

7. The nucleic acid molecule of claim 3 consisting of a fragment of SEQ ID NO:1 having at least 10 nucleotides.

8. The nucleic acid molecule of claim 3 consisting of a fragment of SEQ ID NO:1 having 12-150 nucleotides.

9. The nucleic acid molecule of claim 3 consisting of a fragment of SEQ ID NO:1 having 15-50 nucleotides.

10. An oligonucleotide molecule comprising a nucleotide sequence complementary to a nucleotide sequence of at least 5 nucleotides of SEQ ID NO:1.

11. The oligonucleotide molecule of claim 10 wherein said oligonucleotide molecule comprises a nucleotide sequence complementary to a nucleotide sequence of 5-50 nucleotides of SEQ ID NO:1.

12. The oligonucleotide molecule of claim 10 wherein said oligonucleotide molecule comprises a nucleotide sequence complementary to a nucleotide sequence of 10-40 nucleotides of SEQ ID NO:1.

13. The oligonucleotide molecule of claim 10 consisting of a nucleotide sequence complementary to a nucleotide sequence of at least 10-150 nucleotides of SEQ ID NO:1.

14. The oligonucleotide molecule of claim 10 consisting of a nucleotide sequence complementary to a nucleotide sequence of at least 18-28 nucleotides of SEQ ID NO:1.

15. An isolated antibody which binds to an epitope on SEQ ID NO:2.

16. The antibody of claim 15 wherein said antibody is a monoclonal antibody.

17. A method of identifying modulators of Caspase-1 protein protease activity comprising the steps of:
performing a test assay by contacting a Caspase-1 protease protein with a Caspase-1 substrate in the presence of a test compound, determining the level of processing of said substrate by said protease, and
comparing said level to the level of processing of a Caspase-1 substrate by Caspase-1 protease protein in the absence of said test compound.

18. The method of claim 17 wherein said protein has SEQ ID NO:2.

19. The method of claim 17 wherein said substrate is FKBP46 protein.

20. A method of diagnosing whether a subject has, or is at risk for developing, a cancer, comprising determining the nucleotide sequence of an ARTS1 gene in a sample from the subject, wherein an alteration in the nucleotide sequence, relative to an ARTS1 gene sequence in a control sample, is indicative of the subject having, or being at risk for developing, a cancer.

21. The method of claim 20, wherein the cancer is selected from the group consisting of leukemia, melanoma, lymphoma, myeloma, pancreatic cancer, breast cancer, prostate cancer, colorectal cancer, lung cancer, ovarian cancer, kidney cancer, idiopathic pancytopenia, gastric cancer, Hodgkin's disease, non-Hodgkin's disease, esophogeal cancer, cervical cancer and thyroid cancer.

22. The method of claim 20, wherein the cancer is selected from the group consisting of chronic lymphocytic leukemia (CLL), lung carcinoma, thyroid adenoma, kidney carcinoma, and essential thrombocytemia.

23. The method of claim 20, wherein the cancer is chronic lymphocytic leukemia (CLL).

24. The method of claim 23, wherein the CLL is familial CLL.

25. The method of claim 20, wherein the alteration in the nucleotide sequence is a G to A nucleotide change at position 446 of SEQ ID NO. 15.

26. The method of claim 20, wherein the alteration in the nucleotide sequence is a nucleotide change selected from the group consisting of: a C to T change at position 66 of SEQ ID NO. 15; a C to T change at position 392 of SEQ ID NO. 15; a T to C change at position 442 of SEQ ID NO. 15; and a G to A change at position 490 of SEQ ID NO. 15.

27. The method of claim 20, wherein the alteration is a loss-of-function mutation in the ARTS1 gene.

28. The method of claim 20, wherein the alteration results in the expression of an ARTS1 protein having reduced tumor suppressor activity.

29. The method of claim 20, wherein the alteration results in the expression of a truncated ARTS1 protein.

30. The method of claim 29, wherein the truncated ARTS1 protein is lacking the C-terminal 48 amino acids of SEQ ID NO. 2.

31. A method of diagnosing whether a subject has, or is at risk for developing, a cancer, comprising determining an ARTS1 gene copy number in a sample from the subject, wherein a copy number that is less than two is indicative of the subject having or being at risk for developing a cancer.

32. The method of claim 31, wherein the cancer is selected from the group consisting of leukemia, melanoma, lymphoma, myeloma, pancreatic cancer, breast cancer, prostate cancer, colorectal cancer, lung cancer, ovarian cancer, kidney cancer, idiopathic pancytopenia, gastric cancer, Hodgkin's disease, non-Hodgkin's disease, esophogeal cancer, cervical cancer and thyroid cancer.

33. The method of claim 31, wherein the cancer is selected from the group consisting of chronic lymphocytic leukemia (CLL), lung carcinoma, thyroid adenoma, kidney carcinoma, and essential thrombocytemia.

34. The method of claim 31, wherein the cancer is chronic lymphocytic leukemia (CLL).

35. The method of claim 34, wherein the CLL is familial CLL.

36. The method of claim 31, wherein the copy number is evaluated by loss-of-hyterozogosity (LOH) analysis.

37. The method of claim 36, wherein the LOH analysis is performed using a chromosomal marker that is closely linked to the ARTS1 gene.

38. A method of diagnosing whether a subject has, or is at risk for developing, a cancer, comprising assessing the DNA methylation status of an ARTS1 gene region in a sample from the subject, wherein an increase in the number of methylated nucleotide residues in the ARTS1 gene region, relative to the number of methylated nucleotide residues in a corresponding ARTS1 gene region in a control sample, is indicative of the subject having, or being at risk for developing, a cancer.

39. The method of claim 38, wherein the cancer is selected from the group consisting of leukemia, melanoma, lymphoma, myeloma, pancreatic cancer, breast cancer, prostate cancer, colorectal cancer, lung cancer, ovarian cancer, kidney cancer, idiopathic pancytopenia, gastric cancer, Hodgkin's disease, non-Hodgkin's disease, esophogeal cancer, cervical cancer and thyroid cancer.

40. The method of claim 38, wherein the cancer is selected from the group consisting of chronic lymphocytic leukemia (CLL), lung carcinoma, thyroid adenoma, kidney carcinoma, and essential thrombocytemia.

41. The method of claim 38, wherein the cancer is chronic lymphocytic leukemia (CLL).

42. The method of claim 41, wherein the CLL is familial CLL.

43. The method of claim 38, wherein the ARTS1 gene region comprises SEQ ID NO. 1.

44. The method of claim 38, wherein the ARTS1 gene region comprises nucleotides 1-336 of SEQ ID NO. 1.

45. The method of claim 38, wherein the ARTS1 gene region comprises all or part of the ARTS1 promoter.

46. The method of claim 38, wherein the methylation state is determined using a technique comprising sodium bisulfite conversion and methylation-sensitive PCR.

47. A method of diagnosing whether a subject has, or is at risk for developing, a cancer, comprising assessing the expression level of at least one ARTS1 gene product in a sample from the subject, wherein a decrease in the level of the at least one ARTS1 gene product, relative to the level of a corresponding ARTS1 gene product in a control sample, is indicative of the subject having, or being at risk for developing, a cancer.

48. The method of claim 47, wherein the cancer is selected from the group consisting of leukemia, melanoma, lymphoma, myeloma, pancreatic cancer, breast cancer, prostate cancer, colorectal cancer, lung cancer, ovarian cancer, kidney cancer, idiopathic pancytopenia, gastric cancer, Hodgkin's disease, non-Hodgkin's disease, esophogeal cancer, cervical cancer and thyroid cancer.

49. The method of claim 47, wherein the cancer is selected from the group consisting of chronic lymphocytic leukemia (CLL), lung carcinoma, thyroid adenoma, kidney carcinoma, and essential thrombocytemia.

50. The method of claim 47, wherein the cancer is chronic lymphocytic leukemia (CLL).

51. The method of claim 50, wherein the CLL is familial CLL.

52. The method of claim 47, wherein the cancer is associated with a defective ARTS1 gene.

53. The method of claim 47, wherein the ARTS1 gene product is RNA.

54. The method of claim 47, wherein the ARTS1 gene product is protein.

55. The method of claim 47, wherein the level of the ARTS1 gene product is determined using a method selected from the group consisting of Northern blotting, quantitative or semi-quantitative RT-PCR and Western blotting.

56. A method of preventing or treating a cancer in a subject, comprising administering an effective amount of an ARTS1 gene or gene product to the subject.

57. The method of claim 56, wherein the ARTS1 gene or gene product is introduced into cells of the subject.

58. The method of claim 56, wherein the subject has a defective ARTS1 gene.

59. The method of claim 56, wherein the cancer is selected from the group consisting of leukemia, melanoma, lymphoma, myeloma, pancreatic cancer, breast cancer, prostate cancer, colorectal cancer, lung cancer, ovarian cancer, kidney cancer, idiopathic pancytopenia, gastric cancer, Hodgkin's disease, non-Hodgkin's disease, esophogeal cancer, cervical cancer and thyroid cancer.

60. The method of claim 56, wherein the cancer is selected from the group consisting of chronic lymphocytic leukemia (CLL), lung carcinoma, thyroid adenoma, kidney carcinoma, and essential thrombocytemia.

61. The method of claim 56, wherein the cancer is chronic lymphocytic leukemia (CLL).

62. The method of claim 61, wherein the CLL is familial CLL.

63. The method of claim 56, wherein the ARTS1 gene or gene product is a wild-type gene or gene product.

64. The method of claim 56, wherein the wild-type ARTS1 gene or gene product is an ARTS1 gene.

65. The method of claim 64, wherein the ARTS1 gene comprises the nucleotide sequence depicted in SEQ ID NO 1.

66. The method of claim 65, wherein the ARTS1 gene encodes a polypeptide comprising the amino acid sequence depicted in SEQ ID NO 2, or a biologically-active fragment thereof.

67. The method of claim 65, wherein the wild-type ARTS1 gene is administered via an expression vector.

68. The method of claim 65, wherein the wild-type ARTS1 gene is administered via a targeting vector used for gene replacement therapy.

69. The method of claim 56, wherein the ARTS1 gene or gene product is an ARTS1 gene product.

70. The method of claim 69, wherein the ARTS1 gene product is RNA.

71. The method of claim 69, wherein the ARTS1 gene product is protein.

72. The method of claim 71, wherein the protein comprises the amino acid sequence depicted in SEQ ID NO. 2 or a fragment thereof.
